(19) United States
(12) Patent Application Publication
(10) Pub. No.: US 2010/0111888 A1
(43) Pub. Date: May 6, 2010

(54) Organic Compounds and Compositions Having the Ability to Modulate Fragrance Compositions

(75) Inventors: Boris Schilling, Knonau (CH); Thierry Granier, Duebendorf (CH); Georg Frater, Winterthur (CH); Andreas Hanhart, Uster (CH)

Correspondence Address: PARFOMAK, ANDREW N.;NORRIS MCLAUGHLIN & MARCUS PA, 875 THIRD AVE, 8TH FLOOR, NEW YORK, NY, 10022, US

(73) Assignee: GIVAUDAN SA, Vemier (CH)

(21) Appl. No.: 12/532,790

(22) PCT Filed: Mar. 20, 2008
(86) PCT No.: PCT/CH2008/000128

§ 371 Date: Sep. 23, 2009

(30) Foreign Application Priority Data
Mar. 28, 2007 (GB) 0705944.7

(51) Int. Cl. A61L 9/01; C07C 49/20; A61K 31/12; C07C 49/213; C07C 49/215; C07D 333/02; A61K 31/381; C07D 307/02; A61K 31/34; C07C 49/255; A61K 31/121; C07D 317/10; A61K 31/357; C07D 213/46; A61K 31/44; A61P 43/00

(52) U.S. Cl. 424/76.1; 568/303; 514/690; 568/308; 514/678; 568/328; 514/682; 549/78; 514/438; 549/498; 514/461; 568/415; 514/675; 549/454; 514/467; 546/340; 514/277

(57) ABSTRACT

Disclosed are compounds having the ability to modulate, namely to improve, enhance and or modify fragrance compositions due to their ability to inhibit cytochrome P450 enzymes, e.g. CYP2A13 and CYP2B6.

This invention relates to a class of chemical compounds having the ability to modulate, namely to improve, enhance and or modify fragrance compositions.

The conventional way to create fragrance compositions in the fragrance industry is by the addition of chemical compounds which as such are recognised by a skilled person to possess a positive or pleasant odour themselves. In addition, chemical compounds, to be suitable as fragrances have to fulfil several criteria, for example, a low odour threshold.

Surprisingly there has been found a new class of compounds having the ability to modulate the perception of odorant compounds. Modulators are compounds that influence the olfactive perception of odorant compounds. A modulator may result in changes of intensity (overall enhancer or masking agent), quality (change of olfactive note, enhancing or masking of particular notes), duration/longevity of perception, or combinations thereof. A modulator may also enhance the overall perception of a particular odorant or mixture of odorants, or a particular olfactive quality/note.

It is believed, without being bound by theory, that the modulating effect of the compounds hereinbelow described occurs mainly because of the inhibition of the cytochrome P450 enzyme CYP2A13. This enzyme is predominantly expressed in the human respiratory tract, such as lung tissue, trachea and olfactory mucosa (Su et al., 2000, Cancer Res. 60: 5074-5079). It is known from the art that this enzyme is responsible for the metabolism of a number of chemical compounds, such as coumarin, a well known odorant compound, or 4-(methylnitrosamino)-1-(3-pyridyl)-1-butanone (NNK) a potent tobacco-specific nitrosamine. NNK is formed during the processing and curing of tobacco plants by nitrosation, and it is also believed that nicotine could be converted endogenously to NNK. It is present in tobacco and in tobacco smoke, both mainstream and in sidestream smoke. NNK is a procarcinogen which is metabolically activated by alpha-hydroxylation catalysed by cytochrome P450 activity and the resulting reactive electrophilic metabolites ultimately alkylate DNA.

Cytochrome P450 enzymes constitute a sub-family of heme-thiolate enzymes, which catalyse primarily mono-oxygenase reactions involving a two-stage reduction of molecular oxygen and subsequent single-oxygen atom insertion, although reductive metabolism is also known. Reactions catalysed included hydroxylation, epoxidation, N-oxidation, sulfooxidation, N-, S- and O-dealkylations, desulfation, deamination, and reduction of azo-, nitro- and N-oxide groups. In particular it has been found that most frequently hydroxylation occurs in the presence of CYP2A13, but demethylation of C-methyl and N-methyl, and epoxidation of double bonds also occur. CYP2A13 is dominantly expressed in the human nose and the respiratory tract, however, other P450 enzymes also contribute to metabolism. In particular CYP2A6 and CYP2B6 are prone to metabolize small molecular weight substrates. CYP2B6 also has been identified as being the second important catalyst besides CYP2A13 which is metabolically activating tobacco-specific nitrosamines, such as NNK (Hecht, S. S. (2008) Chem. Res. Toxicol. 21:160-171. Progress and challenges in selected areas of tobacco carcinogenesis). Examples of biochemical reactions catalysed by CYP2A13 are shown in Scheme 1.

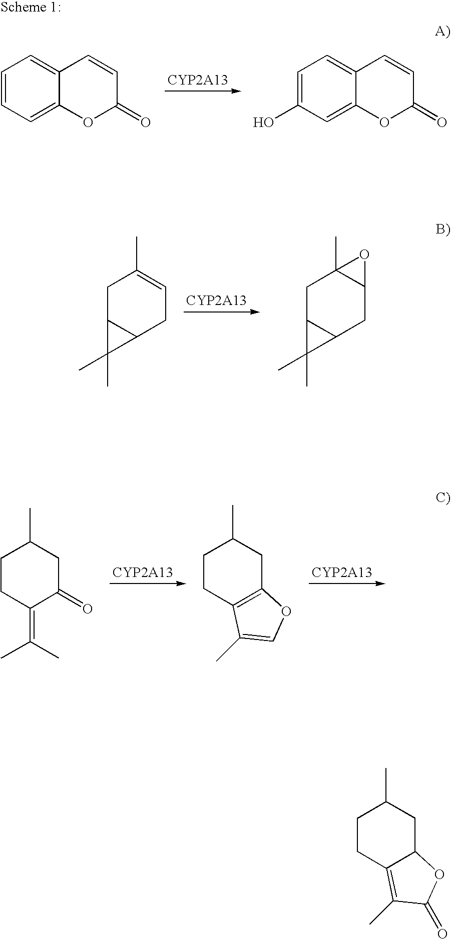

CYP2A13 is one of three members of the human CYP2A family. The other two are CYP2A6 and CYP2A7. Whereas CYP2A6 seems to be a major human liver metabolic enzyme, which also hydroxylates coumarin and metabolises nicotine to cotinine, for CYP2A7 a catalytic activity is presently unknown and it is believed to be a pseudogene. CYP2A6 is also detected in the human respiratory tract, but CYP2A13 is the dominantly expressed isoform.

The metabolism of odorants occurring in the nose may influence olfactory sensation, and respiratory tract metabolism in general, for example in the lung tissue, may influence retronasal olfactory sensation by exchange of air passing though the respiratory tract including the nose, whereby metabolites formed by lung enzymes may reach the olfactory mucosa and receptors located therein. By inhibition of the enzymes responsible for the metabolism, in particular CYP2A13, modulation of the perception of odorant compounds in the nasal cavity can be achieved, as is shown in further detail by the examples.

Accordingly the present invention refers in one of its aspects to a compositions comprising

a) a compound of formula (I)

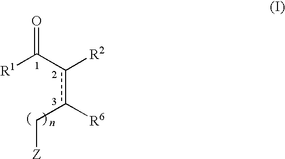

wherein
n is 0 or 1;
the dotted line represents together with the carbon-carbon bond a double bond, either in E or Z configuration, or a single bond;
R¹ is C₁-C₃ alkyl (e.g. ethyl), C₃-C₇ alkenyl (e.g. 3-methyl but-2-en-1yl), cycloalkylvinyl comprising from 5 to 7 carbon atoms (e.g. cyclopropylethenyl), arylvinyl comprising from 5 to 7 carbon atoms (e.g. phenylethylene), phenyl, hydroxyl, C₁-C₃ alkoxy (e.g. methox or ethoxy), or C₂-C₃ alkenyloxy (e.g. —O—CH₂—CH═CH₂), or ethinyl;
R² is linear or branched C₃-C₇ alkyl, such as C₄ alkyl (n-butyl, tert. butyl, 2-methyl-(propyl), but-2-yl), C₅ alkyl (e.g. n-pentyl, 3-methyl(but-1-yl)) and C₆ alkyl (e.g. n-hexyl);
   I) Z is —CR³R⁴R⁵ wherein R³, R⁴, R⁵ are hydrogen; R³ and R⁴ are methyl and R⁵ is hydrogen or methyl; or R³ and R⁴ representing independently H, or C₁-C₆ alkoxy (e.g. ethoxy, propoxy) and R⁵ is C₁-C₆ alkoxy (e.g. ethoxy, propoxy);
   II) Z is a 3-6 membered monocyclic or 6-10 bicyclic hydrocarbon ring (e.g. cyclopropyl, cyclobutyl, cyclopentyl, cyclopentadienyl, cyclopentenyl, cyclohexenyl, cyclohexyl, phenyl, naphtyl) wherein up to two, i.e. 0, 1 or 2, C atom(s) are replaced by a hetero atom selected from S, O, and N (e.g. furanyl, thienyl, tetrahydrofuranyl, benzo-1,3-dioxolyl (e.g. benzo-1,3-dioxo-5-yl), pyridyl, imidazolyl);
   III) Z is a 3-6 membered monocyclic or 6-10 membered bicyclic hydrocarbon ring (e.g. cyclopropyl, cyclobutyl, cyclopentyl, cyclopentadienyl, cyclopentenyl, cyclohexenyl, cyclohexyl, phenyl, naphtyl) wherein up to two, i.e. 0, 1 or 2, C atom(s) are replaced by a hetero atom selected from S, O, and N, and the ring is substituted with up to 5 groups (e.g. 1 or 2 groups) selected from hydroxyl, CN, halogen (e.g. F, Cl, Br), mono-, di-, and trihalogenomethyl (e.g. CF₃), C₁-C₃ alkoxy (e.g. methoxy, ethoxy), C₁-C₃ alkyl (e.g. ethyl), —COOR, and —OCOR wherein R is hydrogen, methyl, ethyl, propyl or isopropyl, with the proviso that the ring is substituted with up to one C₁-C₃ alkyl group only;
   IV) Z is a bivalent residue —CH₂—CH₂— forming together with the C-2 a cyclobutan and cyclopentan ring respectively; or
   V) Z is —C(O)R⁷ wherein R⁷ is C₁-C₃ alkyl (e.g. ethyl, methyl), or C₁-C₃ alkoxy (e.g. ethoxy);
R⁶ is H, C₁-C₃ alkyl (e.g. methyl, ethyl), or —CH₂— forming with C-2 a cyclopropan ring; and
the compound of formula (I) contains at least 9 C-atoms (e.g. 9, 10, 11, 12, 13, 14, 15, 16, 17 C-atoms);

and

b) at least one odorant compound.

The term “odorant compound” as used herein refers to both the volatile part of a flavour and to fragrance molecules. Examples of odorant compounds can be found e.g. in Allured's Flavor and Fragrance Materials 2004, published by Allured Publishing Inc.

Non-limiting examples are compounds of formula (I) wherein R¹ is methyl, R² is selected from n-propyl, n-butyl, n-pentyl, n-hexyl and n-heptyl, R⁶ is hydrogen and Z is cyclopropyl, phenyl, naphthyl, furanyl, thienyl or tetrahydrofuranyl.

Further non-limiting example compounds of formula (I) may be selected from the list of compounds of formula (I) wherein R¹ is methyl, R² is selected from n-propyl, n-butyl, n-pentyl, n-hexyl and n-heptyl, R⁶ is hydrogen and Z is phenyl substituted with one or two groups selected from CN, halogen (e.g. F, Cl, Br), C₁-C₃ alkoxy (e.g. methoxy, ethoxy), C₁-C₃ alkyl and —COOR, wherein R is hydrogen, methyl, ethyl, propyl or is isopropyl.

Further non-limiting examples are compounds of formula (I) wherein R¹ is methyl, R² is selected from n-propyl, n-butyl, n-pentyl, n-hexyl and n-heptyl, and Z is —CR³R⁴R⁵ wherein R³ is hydrogen and R⁴ and R⁵ representing independently C₁-C₆ alkoxy, such as methoxy or ethoxy, or compounds of formula (I) wherein R¹ is methyl, R² is selected from ethyl, n-propyl, n-butyl, n-pentyl, n-hexyl and n-heptyl, and Z is 2-methyl dioxolan-2-yl.

In particular embodiments are compounds of formula (I) selected from the list consisting of (E)-3-(cyclopropylmethylene)octan-2-one; (E)-3-(cyclopropylmethylene)heptan-2-one; (E)-3-(cyclopropylmethylene)nonan-2-one; (1E,4E)-1-cyclopropyl-4-(cyclopropylmethylene)dec-1-en-3-one; (E)-3-benzylideneheptan-2-one; (E)-3-benzylideneoctan-2-one; (1E,4E)-4-benzylidene-1-phenylnon-1-en-3-one; (E)-3-benzylidenenonan-2-one; 3-phenylmethylheptan-2-one; 3-phenylmethyloctan-2-one; (E)-4-(2-acetylhept-1-enyl)-benzonitrile; (E)-3-(naphthalen-2-ylmethylene)octan-2-one; (E)-3-(thiophen-2-ylmethylene)octan-2-one; (E)-3-(furan-2-ylmethylene)octan-2-one; 3-((tetrahydrofuran-2-yl)methyl)octan-2-one; (E)-3-((tetrahydrofuran-3-yl)methylene)heptan-2-one; (E)-3-((tetrahydrofuran-3-yl)methylene)octan-2-one; 3-((tetrahydrofuran-3-yl)methyl)octan-2-one; (E)-3-(2,2-dimethoxyethylidene)heptan-2-one; (E)-3-(2,2-dimethoxyethylidene)-octan-2-one; 3-(2,2-dimethoxyethyl)octan-2-one; 3-(2-methoxyethyl)octan-2-one; (E)-3-(2-(2-methyl-1,3-dioxolan-2-yl)ethylidene)octan-2-one; 3-(2-(2-methyl-1,3-dioxolan-2-yl)ethyl)octan-2-one; 3-pentylheptane-2,6-dione; (E)-3-ethylideneoctan-2-one; 1-(2-methyl-1-pentylcyclopropyl)ethanone; 3-(propan-2-ylidene)octan-2-one; methyl 1-pentylcyclopentanecarboxylate; 1-(1-pentylcyclopentyl)ethanone; (E)-3-(cyclohexylmethylene)octan-2-one; (E)-3-(cyclohex-3-enylmethylene)octan-2-one; (E)-3-(cyclopentylmethylene)octan-2-one; (E)-3-(cyclobutylmethylene)octan-2-one; (E)-3-(2-fluorobenzylidene)octan-2-one; (E)-3-(3-fluorobenzylidene)octan-2-one; (E)-3-(4-fluorobenzylidene)octan-2-one; (E)-3-(2,6-difluorobenzylidene)octan-2-one; (E)-3-(2,4-difluorobenzylidene)octan-2-one; (Z)-3-(3,5-difluorobenzylidene)octan-2-one; (E)-3-(3,5-difluorobenzylidene)octan-2-one; (E)-3-(perfluorobenzylidene)octan-2-one; (E)-3-(2-methylbenzylidene)octan-2-one; (E)-3-(3-methylbenzylidene)octan-2-one; (E)-3-(4-methylbenzylidene)octan-2-one; (E)-3-(2-(trifluoromethyl)benzylidene)octan-2-one; (E)-3-benzylidenehexan-2-one; (E)-3-(2-methoxybenzylidene)octan-2-one; (E)-3-(3-methoxybenzylidene)octan-2-one; (E)-3-(4-methoxybenzylidene)octan-2-one; (E)-3-(4-methoxybenzylidene)heptan-2-one; (E)-3-(4-methoxybenzylidene)hexan-2-one; (E)-3-(benzo[d][1,3]dioxol-5-ylmethylene)octan-2-one; (Z)-methyl 4-(2-acetylhept-1-enyl)benzoate; (E)-methyl 4-(2-acetylhept-1-enyl)benzoate; (E)-3-(thiophen-2-ylmethylene)octan-2-one; (E)-3-(pyridin-2-ylmethylene)octan-2-one; (E)-3-(pyridin-3-ylmethylene)octan-2-one; (E)-3-(pyridin-4-ylmethylene)octan-2-one; (E)-methyl 2-(cyclopropylmethylene)heptanoate; (E)-methyl 2-benzylideneheptanoate; methyl 2-(cyclopropylmethyl)heptanoate; methyl 2-benzylheptanoate; 3-(cyclopropylmethyl)octan-2-one; (E)-3-(1-phenylethylidene)octan-2-one; (E)-2-(cyclopropylmethylene)-1-phenylheptan-1 -one; (E)-methyl 2-(2,2-dimethylpropylidene)heptanoate; (E)-2-(2,2-dimethylpropylidene)heptanoic acid; (E)-3-(2,2-dimethylpropylidene)octan-2-one; (E)-3-(2-methylpropylidene)octan-2-one; (E)-4-(cyclopropylmethylene)nonan-3-one; and (E)-4-benzylidenenonan-3-one.

The compounds of formula (I) may comprise one or more chiral centres and as such may exist as a mixture of stereoisomers, or they may be resolved as isomerically pure forms. Resolving stereoisomers adds to the complexity of manufacture and purification of these compounds, and so it is preferred to use the compounds as mixtures of their stereoisomers simply for economic reasons. However, if it is desired to prepare individual stereoisomers, this may be achieved according to methods known in the art, e.g. preparative HPLC and GC, crystallization or by departing from chiral starting materials, e.g. starting from enantiomerically pure or enriched raw materials from the chiral pool such as terpenoids, and/or by applying stereoselective synthesis.

The compounds according to the present invention improve the performance of fragrances, or suppress or mask the perception of undesired olfactory notes of odorant compounds. By suppressing the formation of an undesired note, such as off-notes, a cleaner overall impression of the odour note can be achieved. In general, compounds of formula (I) modify the olfactive profile of a fragrance accord by altering the composition of odorant compounds that are present in the human nose, and particularly in the olfactory epithelium where they are available to olfactory receptors.

Extensive research has shown that a large number of known odorant compounds undergo a biochemical transformation in the presence of CYP2A13. Accordingly, if an CYP2A enzyme substrate is an odorant compound and the metabolite is an essentially odourless compound, a compound of less intense odor or a compound with a different odor characteristic than the odorant compound itself, then the inhibition of the enzyme will result in a slower reaction of the enzyme with the odorant compound resulting in an intensification of the overall odor or changing particular olfactive notes.

Accordingly, compounds of formula (I) are particularly well suited to be in combination with fragrance molecules that undergo a biotransformation, such as
alcohols, e.g. beta-citronellol, cedrol, Ambrinol (1,2,3,4,4a,5,6,7-Octahydro-2,5,5-trimethyl-2-naphthalenol) and nona-2,6-dienol.
aldehydes and ketones, e.g. octahydro-7-methyl-1,4-methanonaphthalen-6(2H)-one, alpha-ionone, beta-ionone, Cetone V (1-(2,6,6-trimethyl 2-cyclohexen-1-yl) -1,6-heptadien-3-one), alpha damascone, Orivone (4-(1,1-Dimethyl-propyl)-cyclohexanone) and Pulegone (5-methyl-2-(propan-2-ylidene)cyclohexanone).
ethers and acetals, e.g. methyl pamplemousse (1,1-dimethoxy-2,2,5-trimethyl-4-hexene), 1,4-cineole (1,4-epoxy-p-menthane) and rose oxyde (2-(2′-methyl-1′-propenyl)-4-methyltetrahydropyran.
esters and lactones, e.g. methyl N-methyl anthranilate, 3-phenylpropyl acetate, ethyl laiton (8-ethyl-1-oxaspiro[4.5]decan-2-one) and methyl laiton (8-methyl-1-Oxaspiro[4.5]decan-2-one).
macrocycles, e.g. Velvione® (cyclohexadec-5-ene-1-one), Habanolide® (Oxacyclohexadec-12-en-2-one) and Cosmone™ (3-methyl-5-cyclotetradecen-1-one).
heterocycles, e.g. isopropyl quinoline, pyralone (6-(1-methylpropyl)quinoline and 2-isopropyl-4-methylthiazole.
nitriles, e.g. citronellyl nitrile, cumin nitrile (4-(1-methylethyl)-benzonitrile), lemonile (3,7-dimethyl-2,6-Nonadienenitrile), terranile (3-(2,6,6-trimethyl-1-cyclohexen-1-yl)-2-propenenitrile), decanonitrile, and rose nitrile (3-(4,7,7-trimethylbicyclo[4.1.0]hept-3-ylidene)-propanenitrile).
hydrocarbons, e.g. alpha pinene, limonene, terpinolene and delta-3-carene.

Depending on the fragrance composition, there can be achieved by the addition of an effective amount of a compound of formula (I) a completely different odour note compared to a composition not comprising such a modulator. This is further illustrated by the examples.

Inhibitors, namely compounds of formula (I), can be odorants themselves and therefore can contribute to the olfactive profile of a fragrance composition in addition to inhibiting nasal- and/or respiratory tract metabolism. Such inhibitors are preferably used at concentrations at which they are not consciously perceived, namely below their sensory threshold concentration. Accordingly, compounds having a high sensory threshold are preferred; those can be used in higher concentrations without contributing themselves to the olfactive profile of a fragrance accord, while still showing modulatory effects resulting from the inhibition of P450 enzymes, in particular CYP2A13, CYP2A6, and CYP2B6.

The sensory threshold concentration is defined as the concentration of an odorant compound for which the probability of detection of the stimulus is 0.5 (that is 50% above chance, by a given individual, under the condition of the test). The sensory threshold concentration can be measured by standard methods, for example, ASTM E1432-91 and is measured either by olfactometry means or by using sniff-bottles, allowing panellists to smell the presented headspace. It is also possible to smell the presented odour in a sequential process.

Due to the fact that the compounds of formula (I) inhibit the enzyme activity of CYP2A, e.g. CYP2A6 and CYP2A13, and CYP2B6 they may also be used in combination with tobacco products to reduce or inhibit the metabolism of NNK in the respiratory tract when inhaled together with the tobacco smoke.

Accordingly, the present invention refers in a further aspect to a tobacco product, such as cigarettes, chewing tobacco, snuff tobacco, pipes tobacco and cigars, comprising at least one compound of formula (I). If used for tobacco products the addition of about 0.1 to 2% by weight, such as about 0.3 to 1% by weight, e.g. about 1% by weight based on the end product may be sufficient to achieve an effect.

Due to their properties as inhibitors for CYP2A and CYP2B enzymes, they may also be used for the regulation of nicotine metabolism in an individual, such as a nicotine replacement therapy.

Accordingly, the present invention refers in a further of its aspects to the preparation of a pharmaceutical composition comprising a compound of formula (I) as defined hereinabove.

The compounds of the present invention can be administered for, for example, oral, nasal, topical, parenteral, local or inhalant use. Oral administration includes the administration in form of tablets, capsules, chewing gums and sprays.

Furthermore, it is assumed that, if inhaled in the presence of tobacco smoke which comprise NNK, the compounds of formula (I) reduces the NNK metabolic process, because of their properties as inhibitor for CYP2A and/or CYP2B enzymes.

Accordingly, the present invention refers in a further aspect to a method comprising the step of disseminating a compound of formula (I) as defined hereinabove into a room comprising tobacco smoke. Any means capable of disseminating a volatile substance into the atmosphere may be used. The use in this specification of the term “means” includes any type of air-freshener devices which may include a heater and/or fan and nebulization systems well known to the art.

Whereas some compounds of formula (I) are known, others have never been described in literature.

Accordingly, the present invention refers in a further aspect to compounds of formula (Ia)

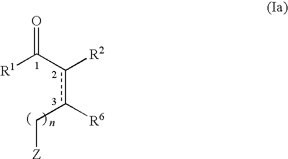

wherein

n is 0 or 1;

the dotted line represents together with the carbon-carbon bond a double bond, either in E or Z configuration, or a single bond;

R¹ is C₁-C₃ alkyl, C₃-C₇ alkenyl (e.g. 3-methyl but-2-en-lyl), cycloalkylvinyl comprising from 5 to 7 carbon atoms (e.g. cyclopropylethenyl), arylvinyl comprising from 5 to 7 carbon atoms (e.g. phenylethylene), phenyl, C₁-C₃ alkoxy (e.g. methox or ethoxy), C₂-C₃ alkenyloxy (e.g. —O—CH₂—CH═CH₂), or ethinyl;

R² is linear or branched C₃-C₇ alkyl, such as C₄ alkyl (n-butyl, tert. butyl, 2-methyl-(propyl), but-2-yl), C₅ alkyl (e.g. n-pentyl, 3-methyl(but-1-yl)) and C₆ alkyl (e.g. n-hexyl);

R⁶ is H, C₁-C₃ alkyl, or —CH₂— forming with C-2 a cyclopropan ring;
I) Z is —CR³R⁴R⁵ wherein R³, R⁴, R⁵ are hydrogen; R³ and R⁴ are methyl and R⁵ is hydrogen or methyl; or R³ and R⁴ representing independently H, or C₁-C₆ alkoxy (e.g. ethoxy) and R⁵ is C₁-C₆ alkoxy (e.g. ethoxy);
   with the proviso that

if n is 0, R² is n-pentyl and R¹ is methyl, Z is not prop-2-yl;
II) Z is a 3-6 membered monocyclic or 6-10 bicyclic hydrocarbon ring (e.g. cyclopropyl, cyclobutyl, cyclopentyl, cyclopentadienyl, cyclopentenyl, cyclohexenyl, cyclohexyl, phenyl, naphtyl) wherein up to two, i.e. 0, 1 or 2, C atom(s) are replaced by a hetero atom selected from S, O, and N (e.g. furanyl, thienyl, tetrahydrofuranyl, benzo-1,3-dioxolyl (e.g. benzo-1,3-dioxo-5-yl), pyridyl, imidazolyl);
   with the proviso that

if n is 0, R² is [n-pentyl] linear C₃-C₅ alkyl and R¹ is methyl, Z is not phenyl;

if n is 0, R² is linear C₃-C₄ alkyl and R¹ is methyl, Z is not methoxyphenyl;

if n is 0, R² is n-pentyl and R¹ is methoxy, Z is not phenyl;

if n is 0, R² is n-hexyl, R¹ is methyl, and the carbon-carbon bond between C-2 and C-3 is a single bond, Z is not cyclopropyl;
III) Z is a 3-6 membered mono- or bicyclic hydrocarbon ring (e.g. cyclopropyl, cyclobutyl, cyclopentyl, cyclopentadienyl, cyclopentenyl, cyclohexenyl, cyclohexyl, phenyl, naphtyl) wherein up to two, i.e. 0, 1 or 2, C atom(s) are replaced by a hetero atom selected from S, O, and N, and the ring is substituted with up to 5 (e.g. 1 or 2 groups) groups selected from hydroxyl, CN, halogen (e.g. F, Cl, Br), mono-, di-, and trihalogenomethyl (e.g. CF₃), C₁-C₃ alkoxy (e.g. methoxy, ethoxy), C₁-C₃ alkyl, —COOR and —OCOR, wherein R is hydrogen, methyl, ethyl, propyl or isopropyl, with the proviso that the ring is substituted with up to one C₁-C₃ alkyl group;
IV) Z is a bivalent residue —CH₂—CH₂— forming together with the C-2 a cyclobutan and cyclopentan ring respectively; or
V) Z is —C(O)R⁷ wherein R⁷ is C₁-C₃ alkyl, or C₁-C₃ alkoxy;

and the compound of formula (Ia) contains at least 9 C-atoms (e.g. 9, 10, 11, 12, 13, 14, 15, 16, 17 C-atoms);

with the proviso that the compound of formula (Ia) is not 3-ethylideneoctan-2-one or 3-(propan-2-ylidene)octan-2-one.

Compounds of formula (Ib), i.e. compound of formula (I) wherein R⁶ is hydrogen, may be prepared by Wittig-Horner reaction of the appropriate aldehyde (4) with an appropriate acyl phosphonate (3) synthesized in two steps via a first phosphonate (2), obtained by Arbuzov reaction of an appropriate alkyl iodide (1) with triethyl phosphite, by deprotonation and acylation, as shown in scheme 2 (wherein R¹, R² and Z have the same meaning as given in the description above for formula (I)).

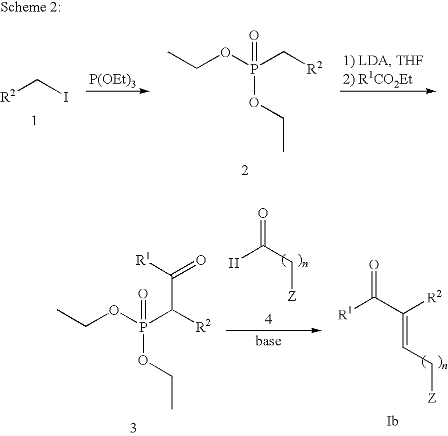

A more direct way consists in the condensation of methyl ketones with aldehydes as shown in scheme 3.

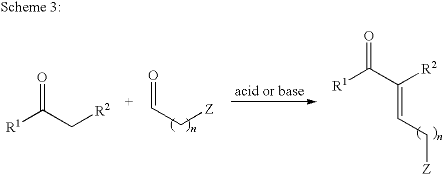

Tetrasubstitued olefins, i.e. compound of formula (Ic) wherein R⁶ is not hydrogen, may be prepared by alkylation of the appropriated oxide (5) by isomerization, under conditions known to the person skilled in the art, as depicted in scheme 4 (R¹, R², R⁶ and Z have the same meaning as given above for formula (I)).

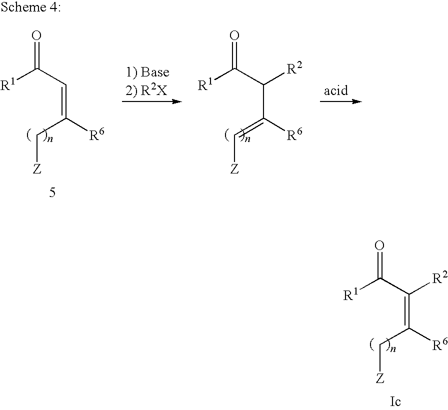

A further option to prepare the tetrasubstitued olefins consists in the reaction of silyl enol ethers with alkynes as shown in scheme 5 below.

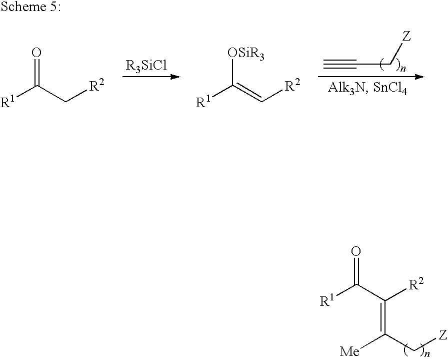

The invention is now further described with reference to the following non-limiting examples. These examples are for the purpose of illustration only and it is understood that variations and modifications can be made by one skilled in the art.

### EXAMPLE 1

### Evaluation of the Test Compounds as Inhibitors of CYP2A13

Compounds that inhibit the activity of CYP2A13 are identified by using a standard reaction established for the enzyme. A known substrate is coumarin, and the product of the enzymatic reaction is 7-hydroxy-coumarin (Umbelliferone) which is strongly fluorescent. When a compound is added to the standard reaction and the formation of Umbelliferone is decreased, the compound is identified as an inhibitor, which can also be a competitive substrate of the enzyme. The compound is used at various concentrations and the concentration-dependent decrease in Umbelliferone formation allows to determine the concentration where the activity of the enzyme is reduced to the 50% level (IC50 value).

A test compound (details see Table 1) was incubated with CYP2A13 in the presence of a cytochrome P450 reductase. CYP2A13 and P450 reductase were employed in form of microsomes. CYP2A13 was produced in Sf9 cells using a recombinant baculovirus, under conditions known to the person skilled in the art, for example, as described in WO 2006/007751. P450 reductase is commercially available (BD Biosciences Gentest, USA). Preferably, the two enzymes are coexpressed in the same insect cells and microsomes prepared which contain both enzymes. The art of coexpression of two enzymes is known, and the coexpression CYP2A13 and P450 reductase is described in WO 2006/007751. Variability of activity was observed for high-titer recombinant virus batches, and optimal multiplicity of infection (MOI) has to be determined as known to the skilled person. An MOI of 4 for recombinant CYP2A13 baculovirus combined with an MOI of 3.5 for recombinant P450 reductase baculovirus routinely produced microsomes with considerable activity.

Microsomes were used which contained 7 pmoles CYP2A13. Tris buffer (1 M, pH 7.6) and water were added to give a buffer concentration of 0.1 M. The test compound was prepared as a 50 mM stock solution in acetonitrile. The concentration of the standard substrate coumarin was 0.006 mM. Several samples of the test compound were prepared at various concentrations to give different final concentrations in the reaction: 0, 0.005, 0.01, 0.02, 0.05, 0.1 and 0.2 mM. (As obvious to the person skilled in the art, in cases where very good inhibitors were tested, lower concentrations were also used in order to have concentrations above and below the IC50 concentration present in the test wells.) The mixture was incubated for 10 min at 37° C. prior to the initiation of the enzymatic reaction by the addition of 0.005 ml of a solution of 50 mM NADPH in water. The final total volume was 0.2 ml, which is suitable for microtiter plate measurements. The samples were incubated for 60 min at 37° C. After 60 min, the enzymatic reaction was stopped by the addition of 0.02 ml cold 50% trichloroacetic acid (TCA) and incubated at 4° C. for 15 min. 0.005 ml of a solution of 50 mM NADPH in water was added to the control reaction which corresponds to the reaction without test compound and without NADPH, and as a consequence, no Umbelliferone was formed. Denatured proteins and other insoluble parts were separated by centrifugation (10 min, 560×g, room-temperature).

The samples were analysed spectrofluorometrically which allows to detect the formation of Umbelliferone as the enzymatic product of coumarin at an excitation wavelength of 340 nm and an emission wavelength of 480 nm. A decrease of the fluorescent signal at 480 nm with respect to the control shows that the test compound is influencing enzymatic activity and confirms the nature of an inhibitor, since no metabolites have been detected. Graphical analysis of the data allows to calculate the concentration, where the test compound inhibits the enzyme to the level of 50% maximal activity (IC50 value).

TABLE 1
CYP2A13 inhibitor activity

| Compound | |
|---|---|
| IC₅₀ (μM) | Chemical Structure |
| ID 1 0.5 μM | 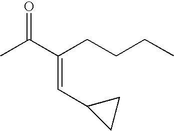 |
| ID 2 1.1 μM | 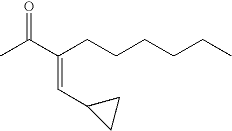 |
| ID 3 0.3 μM | 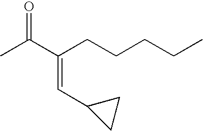 |
| ID 4 4.8 μM | 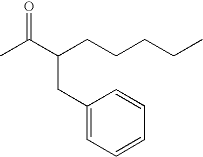 |
| ID 5 0.6 μM | 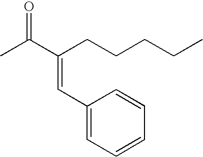 |
| ID 6 3.0 μM | 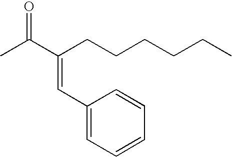 |
| ID 7 2.0 μM | 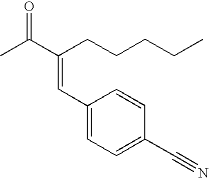 |
| ID 8 16.3 μM | 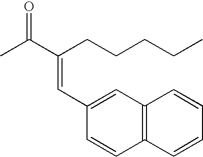 |
| ID 9 5.3 μM | 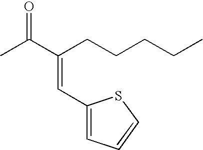 |
| ID 10 15.5 μM | 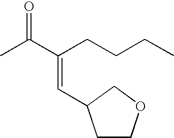 |
| ID 11 1.6 μM | 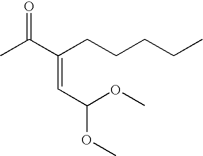 |
| ID 12 3.2 μM | 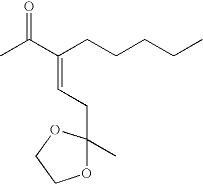 |
| ID 13 9.7 μM | 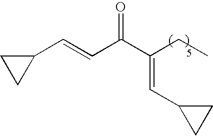 |
| ID 14 2.1 μM | 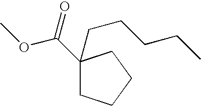 |
| ID 15 0.7 μM | 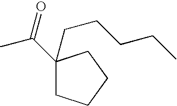 |
| ID 16 0.5 μM | 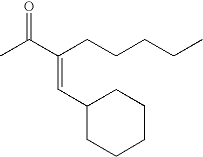 |
| ID 17 0.6 μM | 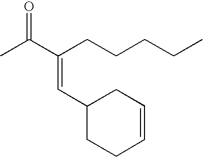 |
| ID 18 0.5 μM | 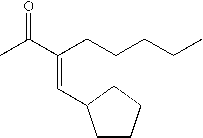 |
| ID 19 0.3 μM | 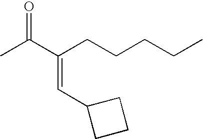 |
| ID 20 0.3 μM | 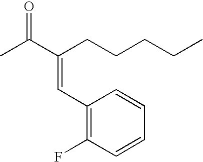 |
| ID 21 0.3 μM | 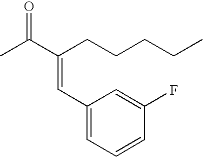 |
| ID 22 0.4 μM | 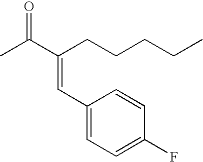 |
| ID 23 0.2 μM | 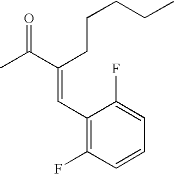 |
| ID 24 0.2 μM | 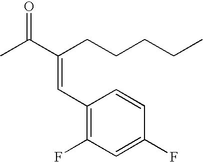 |
| ID 25 0.6 μM | 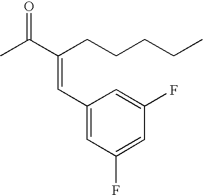 |
| ID 26 14.3 μM | 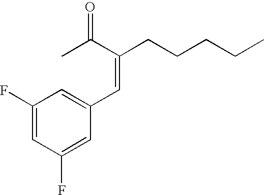 |
| ID 27 0.7 μM | 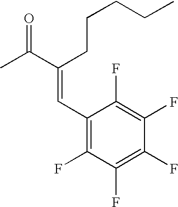 |
| ID 28 0.3 μM | 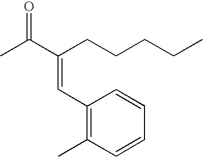 |
| ID 29 0.4 μM | 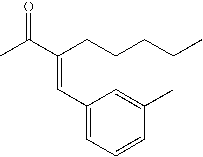 |
| ID 30 0.4 μM | 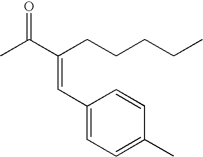 |
| ID 31 0.2 μM | 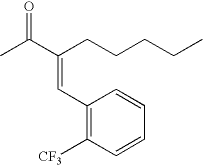 |
| ID 32 1.0 μM | 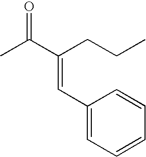 |
| ID 33 0.6 μM | 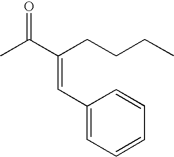 |
| ID 34 0.4 μM | 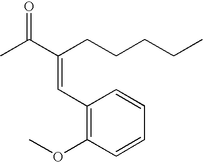 |
| ID 35 0.5 μM | 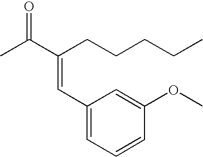 |
| ID 36 0.4 μM | 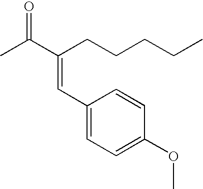 |
| ID 37 1.4 μM | 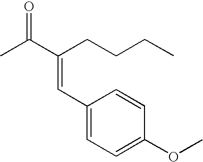 |
| ID 38 2.0 μM | 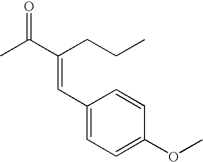 |
| ID 39 2.0 μM | 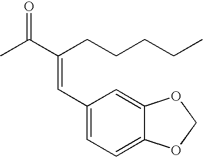 |
| ID 40 1.1 μM | 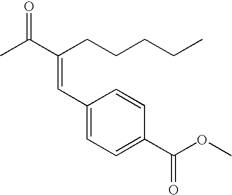 |
| ID 41 47.2 μM | 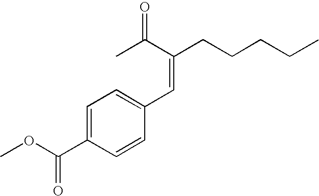 |
| ID 42 0.4 μM | 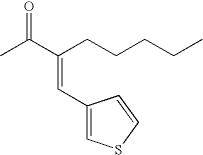 |
| ID 43 0.1 μM | 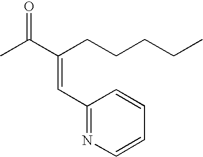 |
| ID 44 0.1 μM | 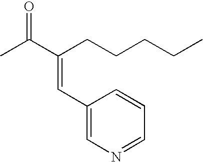 |
| ID 45 0.5 μM | 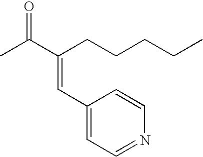 |
| ID 46 2.3 μM | 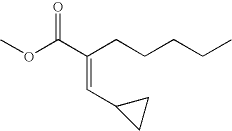 |
| ID 47 0.5 μM | 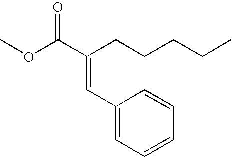 |
| ID 48 8.7 μM | 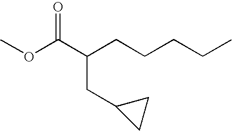 |
| ID 49 4.4 μM | 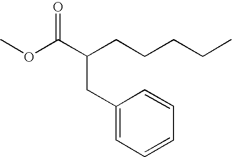 |
| ID 50 1.3 μM | 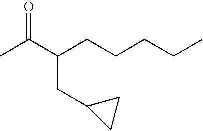 |
| ID 51 2.5 μM | 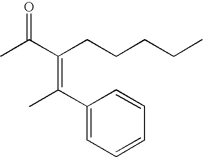 |
| ID 52 6.6 μM | 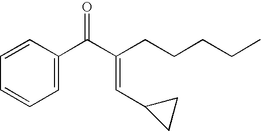 |
| ID 53 1.9 μM | 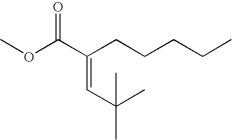 |
| ID 54 2.6 μM | 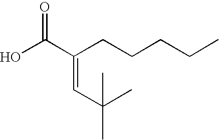 |
| ID 55 0.3 μM | 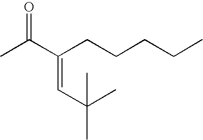 |
| ID 56 0.2 μM | 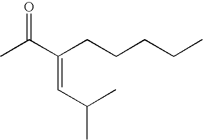 |
| ID 57 0.3 μM | 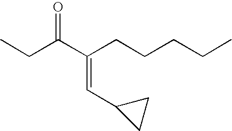 |
| ID 58 0.5 μM | 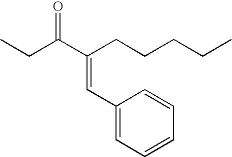 |

Low IC50 values mean that the test compound is a very efficient inhibitor of the enzyme, and for application purposes, where modulating effects are desired, inhibitors with a low IC50 value (e.g. below 5) may be preferred depending on the olfactory threshold of the compound.

### EXAMPLE 2

### Evaluation of the Test Compounds as Inhibitors of CYP2A6

Test compounds that inhibit the activity of CYP2A6 are identified by using the same principle as described in Example 1, first paragraph.

A test compound (details see Table 2) was incubated with CYP2A6 in the presence of a cytochrome P450 reductase. CYP2A6 and P450 reductase were employed in form of microsomes (BD Biosciences Gentest, USA). Microsomes were used which contained 2 pmoles CYP2A6 and an amount of NADPH-P450 reductase corresponding to cytochrome c reductase activity of 87 nmole/(min×mg protein). Tris buffer (Tris-(hydroxymethyl)aminomethane, 1 M, pH 7.6) and water were added to give a buffer concentration of 0.1M. The test compound was prepared as a 50 mM stock solution in acetonitrile. The concentration of of the standard substrate coumarin was 0.003 mM.

Several samples of the test compound were prepared at various concentrations to give different final concentrations in the reaction: 0, 0.005, 0.01, 0.02, 0.05, 0.1 and 0.2 mM. The mixture was incubated for 10 min at 37° C. prior to the initiation of the enzymatic reaction by the addition of 0.005 ml of a solution of 50 mM NADPH in water. The final total volume was 0.2 ml, which is suitable for microtiter plate measurements. The samples were incubated for 60 min at 37° C. After 60 min, the enzymatic reaction was stopped by the addition of 0.02 ml cold 50% trichloroacetic acid (TCA) and incubated at 4° C. for 15 min. 0.005 ml of a solution of 50 mM NADPH in water was added to the control reaction which corresponds to the reaction without test compound and without NADPH, and as a consequence, no Umbelliferone was formed. Denatured proteins and other insoluble parts were separated by centrifugation (10 min, 560×g, room-temperature).

The samples were analysed spectrofluorometrically according to the procedure described in Example 1.

TABLE 2
CYP2A6 inhibitor activity

| Compound | |
|---|---|
| IC₅₀ (μM) | Chemical Structure |
| ID 1 3 μM | 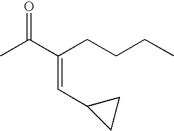 |
| ID 2 8 μM | 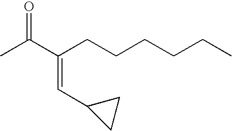 |
| ID 3 7 μM | 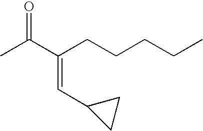 |
| ID 4 148 μM | 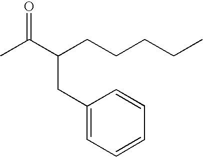 |
| ID 5 141 μM | 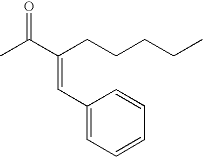 |

### EXAMPLE 3

### Modulation of an Odorant Compound in the Presence of a CYP2A Inhibitor

For demonstration purposes, a simple olfactometer was used as a device to deliver the scent from an odorant compound in the presence/absence of the inhibitor. A dispensing device as described in WO 2004/009142 was used. A cassette with 3 channels was used to release headspace of the “channel 1” containing an odorant compound “A”, “channel 2” containing the inhibitor, i.e. a compound of formula (I), and “channel 3” empty.

As odorant compound “A” 5-isopropenyl 4,8-dimethylbicyclo[3.3.1]non-7-en-2-one was used, which is described as being woody, fruity, raspberry. During enzymatic analysis of the odorant compound “A” with CYP2A13, it has been found that a metabolite “B”, i.e. 5-(3-hydroxyprop-1-en-2-yl)-4,8-dimethylbicyclo[3.3.1]non-7-en-2-one, was produced by the enzyme, which has a very strong raspberry note with a sensory threshold which is 10-times lower than the threshold of “A”. When “A” is reaching the nasal cavity, it is possible that CYP2A13 which is present in the olfactory epithelium is also oxidizing the substrate to “B” which has an intense raspberry smell.

A sensory study was conducted using the above described dispensing device, where “A” was smelled in the presence or absence of the inhibitor compound ID 3 (3-(cyclopropylmethylene)octan-2-one). The odorant “A” was used at a concentration that is rated as pleasant by the panelist, and clearly above threshold. For this purpose, 10 mg of the odorant “A” was dissolved in 10 μl ethanol, and 10 μl loaded in the reservoir of “channel 1” in the cassette. A volume of 10 μl of the inhibitor was used which result in a headspace concentration which was not be perceived as odorous upon activation of the channel and smelling the dispensed ingredient.

When smelling the dispensed odorant compound “A” upon activation of “channel 1”, the panelists described “A” as woody and raspberry. Activation of either “channel 2” which contained the inhibitor, or “channel 3” which was empty, was reported as being odorless. Upon combination of “channel 1” and “channel 2” at the same time, panelists reported that the raspberry aspect was reduced or completely lost. This phenomenon was not observed when combining “channel 1” and “channel 3”.

This result indicates that the odorant compound “A” is predominantly woody and the perception of the raspberry note is mainly derived from the metabolite “B” which is formed enzymatically in the olfactory epithelium. Upon addition of the inhibitor, the formation of “B” is reduced, and as anticipated, the quality of odorant “A” is changed.

Accordingly, if an odorant compound is a substrate of nasal enzymes, in particularly CYP2A13, the combination with an inhibitor as defined by formula (I) will change the olfactive quality of the odorant compound or mixtures thereof.

### EXAMPLE 4

### Modulation of a Fragrance Accord in the Presence of a CYP2A Inhibitor

Two fragrance accords each consisting of 10 ingredients which have been selected in order to demonstrate an odor-modulating effect by the inhibitor. For each panelist, the inhibitor was tested by itself and confirmed that it was rated as being odorless at the given concentration.

Fragrance Accord 1:

| | Parts by |
|---|---|
| | weight 1/900 |
| Benzyl-salicylate | 295 |
| Sandela ® | 200 |
| (3-(5,5,6-trimethylbicyclo[2.2.1]hept-2-yl)-cyclohexan-1-ol) | |
| Thibetolide (oxacyclohexadecan-2-one) | 140 |
| Super muguet (6-ethyl-3-methyl-6-octen-1-ol) | 90 |
| Epoxy cedrene | 70 |
| (octahydro-3,6,6,7a-tetramethyl-2H-2a,7- | |
| Methanoazuleno[5,6-b]oxirene) | |
| Eugenol | 50 |
| Grisalva (naphtho [2,1-b]-furan, 3a-ethyl dodecahydro- | 15 |
| 6,6,9a-trimethyl) | |
| Cis-3-hexenyl-acetate | 15 |
| Beta-damascone | 15 |
| Javanol ® | 10 |
| (1-methyl-2-(1,2,2-trimethylbicyclo[3.1.0]-hex- | |
| 3-ylmethyl)cyclopropyl)methanol) | |

Fragrance Accord 2:

| | Parts by |
|---|---|
| | weight 1/900 |
| Benzyl-salicylate | 305 |
| Ethyl-safranate (ethyl 2,6,6-trimethylcyclohex- | 150 |
| 3-enecarboxylate) | |
| Rosaphen (beta-methyl benzenepentanol) | 100 |
| Musk ketone (4′-tert-butyl-2′,6′-dimethyl- | 80 |
| 3′,5′-dinitroacetophenone) | |
| Propyl diantilis (2-ethoxy-4-(isopropoxymethyl)phenol) | 80 |
| Ebanol ® | 60 |
| (3-methyl-5-(2,2,3-trimethyl-3-cyclopenten-1-yl)- | |
| 4-penten-2-ol) | |
| Beta-damascone | 45 |
| Muscone (3-methyl-cyclopentadecanone) | 40 |
| Grisalva | 30 |
| Cis-3-hexenyl-acetate | 10 |

Sensory studies are performed using an olfactometer, such as the Virtual Aroma Synthesizer (VAS) which is described in Chimia (2001) 55:401-405. The instrument allows to combine saturated headspace of different samples from different containers at various dilutions in order to determine the effect on the odor of the mixture produced in headspace. For the particular example, in one container fragrance accord 1 and 2 respectively (1 gram) was adsorbed on beads (4 grams) and in another container the inhibitor compound ID 3 (1 gram) was adsorbed on beads (4 grams).

Panelists were selected having different levels of experience and expertise in smelling, rating, describing and evaluating odorants, accords and perfumes. Panelists smelled the accords with or without the inhibitor at random order, not knowing which one was presented. Before and after the session, it was confirmed that the inhibitor alone was odorless. Panelists were allowed to select a concentration of the accord that had a pleasant intensity.

The panelist reported an effect that was attributed to the presence of the inhibitor, independent of the experience with perfumery raw materials. The effect was described for both accords as intensifying or boosting the fruitiness of the accord.

In conclusion the example demonstrates that the use of an ingredient which has been identified as an inhibitor of the nasal CYP2A13 can modulate the olfactive quality of a fragrance accord.

### EXAMPLE 5

### Inhibition of NNK Metabolism

The catalytic activity of CYP2A13 in the presence or absence of an inhibitor according to the present invention was tested using radiolabeled [5-³H]NNK as the substrate according to the protocol described in Zhang et al. (2002) J. Pharmacol. Exp. Therap. 302: 416-423, also using NNK from Chemsyn Science Laboratories (Lenexa, Kans., USA).

Two metabolites, keto aldehyde (4-(3-pyridyl)-4-oxobutanal) and keto alcohol (4-hydroxy-1-((3-pyridyl)-1-butanone), which are formed from [5-³H]NNK by a CYP2A13-dependent α-carbon hydroxylation pathway can be detected by high-pressure liquid chromatography with an on-line radioactivity detector.

Procedure: Reaction mixtures contained 100 mM sodium phosphate, pH 7.4, 1 mM EDTA, an NADPH-generating system (5 mM glucose 6-phosphate, 3 mM MgCl₂, 1 mM NADPH, and 1.5 units of glucose-6-phosphate dehydrogenase), 10 μM NNK (containing 1 μCi [5-³H]NNK), 5 mM sodium bisulfite, and 10 pmol of purified, reconstituted CYP2A13 in a total volume of 0.4 ml. CYP2A13 was reconstituted with rat NADPH-P450 reductase, at a ratio of 1:4 (P450/reductase). The inhibitor (compound ID 3) was diluted to 50 mM in acetonitrile based on molecular weight and further diluted to 400 μM by adding 1.2 μl to 148.8 μl water. This concentration was used to reach the final reaction concentrations (10 μl was added for 10 μM and 1 μl was added for 1 μM). The final concentration of acetonitrile was 0.02% in the 10 μM reactions and 0.002% in the 1 μM reactions. Reactions were carried out for 10 minutes at 37° C. before being terminated with 50 μl each saturated barium hydroxide and 25% zinc sulfate. The results are shown in Table 3 below.

TABLE 3
Blocking of the metabolic activation of NNK

| | Keto aldeyde | Keto alcohol |
|---|---|---|
| Inhibitor/controls | (pmol/min/pmol CYP) | (pmol/min/pmol CYP) |
| No inhibitor | 3.46 | 1.33 |
| 1 μM Compound ID3 | N.D. | N.D. |
| 0.002% acetonitrile | 3.28 | 1.06 |
| 10 μM Compound ID 3 | N.D. | N.D. |
| 0.02% acetonitrile | 3.05 | 0.95 |

(N.D. = none detected)

The inhibition results clearly demonstrate that inhibitor, i.e. a compound of formula (I) is an efficient inhibitor of CYP2A13 with an IC50 value clearly below 1 μM for NNK as substrate, since at 1 μM the enzyme was completely inhibited. Acetonitrile which was used as a solvent slightly affects the activity of CYP2A13 at the concentrations used in the enzymatic assay.

### EXAMPLE 6

### (E)-3-(cyclopropylmethylene)octan-2-one (Compound ID 3)

A solution of hexyl iodide (90 ml, 592 mmol) in triethyl phosphite (434 ml, 2.37 mol) was heated for 8 h at 150° C. The reaction mixture was then cooled to 20° C. and distilled using a Vigreux-distillation apparatus (11 mbar, bath temperature: 140-160° C.) giving diethyl hexylphosphonate (111.4 g, 85%). Boiling point: 126° C. (11 mbar).

¹³C-NMR (100MHz, CDCl₃): δ 61.16 (t, J=6.6, 2 CH₂O), 31.14 (t, J=1.0, C(4)), 30.13 (t, J=16.6, C(3)), 25.57 (t, J=140.1, C(1)), 22.25 (t, C(5)), 22.23 (t, J=5.0, C(2)), 16.32 (q, J=5.8, 2 MeCH₂O), 13.83 (q, C(6)).

At −60° C., a solution of diisopropylamine (72.6 ml, 72%, 0.515 mol) in tetrahydrofuran (250 ml) was treated within 15 min. with a 1.6M solution of n-butyllithium in hexane (322 ml, 0.515 mol). The resulting solution was stirred 20 min. at −72° C. and treated with a solution of previously prepared diethyl hexylphosphonate (57.2 g, 0.257 mol) in tetrahydrofuran (150 ml). The resulting solution was stirred for 1 h at −72° C. and treated with a solution of ethyl acetate (37.8 ml, 0.386 mol) in tetrahydrofuran (100 ml). After stirring for 1 h at −70° C., the cooling bath was removed and the solution stirred for 1 h before being diluted with methyl t-butyl ether (250 ml) and acidified with aqueous 2M HCl (200 ml), aqueous 6M HCl (100 ml), and concentrated HCl to pH 6.4. The aqueous phase was extracted with methyl t-butyl ether (200 ml) and the combined organic phases were washed with aqueous NaCl solution (200 ml), dried (Na₂SO₄) and the solvent evaporated. Short-path Vigreux-distillation (0.07 mbar) of the crude product (74.3 g) gave diethyl 2-oxooctan-3-ylphosphonate (52.2 g, 77%). Boiling point: 107° C. (0.07 mbar).

¹³C-NMR (100MHz, CDCl₃): δ 203.90 (s, J=4.1, CO), 62.56 (t, J=6.6, CH₂O), 62.47 (t, J=6.6, CH₂O), 53.75 (d, J=124.4, C(3)), 31.43 (t, C(6)), 31.08 (q, C(1)), 28.19 (t, J=14.9, C(5)), 26.39 (t, J=5.0, C(4)), 22.29 (t, C(7)), 16.34 (q, J=1.7, MeCH₂O), 16.33 (q, J=1.7, MeCH₂O), 13.91 (q, C(8)).

At 4° C., a mixture of a solution of NaOH (12.8 g, 0.32 mol) in water (27 ml) and dichloromethane (100 ml) was treated dropwise with a solution of diethyl 2-oxooctan-3-ylphosphonate (17.0 g, 64.3 mmol) and cyclopropanecarboxaldehyde (4.9 ml, 64.3 mmol) in dichloromethane (20 ml). The resulting mixture was stirred for 89 h at 20° C. and poured into ice/2M aqueous HCl (300 ml). The aqueous phase was extracted with cyclohexane (100 ml). The combined organic phases were washed twice with water (100 ml), dried (Na₂SO₄), and the solvent evaporated. Flash chromatography (FC) (700 g SiO₂, hexane/methyl t-butyl ether 25:1) of the crude product (12.7 g) gave (E)-3-(cyclopropylmethylene)octan-2-one (6.25 g, 54%). Boiling point: 85° C. (0.08 mbar).

¹H-NMR (400 MHz, CDCl₃): δ 5.92 (d, J=10.4, H—C═C(3)), 2.38 (br. t, J=7.6, 2H—C(4)), 2.23 (s, C(1)H₃), 1.75-1.65 (m, H—CCH═), 1.43-1.25 (m, C(5)H₂, C(6)H₂, C(7)H₂), 1.01 (ddd, J=4.3, 6.6, 7.8, 2H), 0.88 (t, J=7.0, C(8)H₃), 0.63 (ddd, J=4.4, 6.5, 8.8, 2H). ¹³C-NMR (100 MHz, CDCl₃): δ 198.51 (s, C(2)), 148.99 (d, CH═C(3)), 140.51 (s, C(3)), 31.89 (t), 29.09 (t), 25.59 (t), 25.36 (q, C(1)), 22.54 (t), 14.01 (q, C(8)), 11.75 (d), 8.74 (t, 2 C). MS (EI): 180 (1), 165 (19), 152 (27), 137 (6), 123 (12), 109 (24), 96 (40), 81 (25), 67 (17), 43 (100). IR: νₘₐₓ 3007, 2956, 2928, 2859, 1659, 1632, 1457, 1392, 1357, 1262, 1174, 1123, 1049, 1022, 986, 954, 939, 847, 808, 722 cm⁻¹.

### EXAMPLE 7

### (E)-3-(cyclopropylmethylene)heptan-2-one (Compound ID 1)

Prepared as described in Example 6 in 38% yield from cyclopropanecarboxaldehyde and diethyl 2-oxoheptan-3-ylphosphonate (obtained from pentyl iodide and triethyl phosphite via diethyl pentylphosphonate). Boiling point: 50° C. (0.09 mbar).

¹H-NMR (400 MHz, CDCl₃): δ 5.92 (d, J=10.4, H—C═C(3)), 2.39 (br. t, J=7.5, 2H—C(4)), 2.24 (s, C(1)H₃), 1.75-1.65 (m, H—CCH═), 1.41-1.29 (m, C(5)H₂, C(6)H₂), 1.01 (ddd, J=4.3, 6.6, 7.8, 2H), 0.91 (t, J=7.3, C(7)H₃), 1.01 (dt, J=4.6, 6.6, 2H). MS (EI): 166 (1), 151 (16), 138 (26), 123 (10), 109 (16), 96 (37), 95 (38), 81 (31), 67 (21), 53 (11), 43 (100).

### EXAMPLE 8

### (E)-3-(cyclopropylmethylene)nonan-2-one (Compound ID 2) and (1E,4E)-1-cyclopropyl-4-(cyclopropylmethylene)dec-1-en-3-one (Compound ID 13)

At 0° C., a mixture of a solution of NaOH (14.3 g, 0.36 mol) in water (22 ml) and dichloromethane (50 ml) was treated dropwise with a solution of diethyl 2-oxononan-3-ylphosphonate (obtained from heptyl iodide and triethyl phosphite via diethyl heptylphosphonate as described in Example 6, 19.9 g, 71 mmol) and cyclopropane-carboxaldehyde (4.9 ml, 64.3 mmol). The resulting mixture was stirred for 15 h at 20° C. and poured into ice/2M aqueous HCl. The aqueous phase was extracted three times with diethyl ether. The combined organic phases were washed with water, dried (Na₂SO₄), and the solvent evaporated. Flash chromatography (FC) (700 g SiO₂, hexane/methyl t-butyl ether 25:1) of the crude product (14.1 g) gave (1E,4E)-1-cyclopropyl-4-(cyclopropylmethylene)dec-1-en-3-one (0.8 g, 5%) and (E)-3-(cyclopropylmethylene)nonan-2-one (2.3 g, 17%).

(E)-3-(cyclopropylmethylene)nonan-2-one (Boiling point: 87° C. at 0.08 mbar):

¹³C-NMR (100 MHz, CDCl₃): δ 198.55 (s, C(2)), 148.99 (d, CH═C(3)), 140.52 (s, C(3)), 31.72 (t), 29.39 (t, 2 C), 25.66 (t), 25.37 (q, C(1)), 22.62 (t), 14.05 (q, C(9)), 11.76 (d), 8.75 (t, 2 C). MS (EI): 194 (1), 179 (12), 166 (17), 151 (5), 137 (5), 124 (6), 123 (16), 109 (35), 96 (60), 81 (29), 67 (21), 43 (100).

(1E,4E)-1-cyclopropyl-4-(cyclopropylmethylene)dec-1-en-3-one (Boiling point: 200° C. at 0.08 mbar):

¹³C-NMR (100 MHz, CDCl₃): δ 190.26 (s, C(3)), 151.84 (d), 147.27 (d), 140.58 (s, C(4)), 122.36 (d), 31.71 (t), 29.36 (t), 29.32 (t), 26.29 (t), 22.61 (t), 14.86 (d), 14.07 (q, C(10)), 11.78 (d), 8.74 (t, 2 C), 8.29 (t, 2 C). MS (EI): 246 (2), 231 (3), 218 (5), 217 (5), 190 (13), 189 (13), 175 (10), 161 (22), 147 (56), 133 (71), 119 (15), 107 (27), 105 (32), 95 (47), 91 (46), 79 (44), 81 (30), 67 (100), 55 (49), 41 (95).

### EXAMPLE 9

### (E)-3-benzylideneheptan-2-one (Compound ID 33)

As described in Example 6, the reaction of benzaldehyde and diethyl 2-oxoheptan-3-ylphosphonate (obtained from pentyl iodide and triethyl phosphite via diethyl pentylphosphonate) in 2:5 water/dichloromethane gave after FC, (E)-3-benzylideneheptan-2-one (22%) and (1E,4E)-4-benzylidene-1-phenyloct-1-en-3-one (19%). Boiling point: 90° C. (0.09 mbar).

¹H-NMR (400 MHz, CDCl₃): δ 7.47 (s, H—C═C(3)), 7.45-7.31 (m, 5H), 2.53-2.47 (m, 2H—C(4)), 2.45 (s, C(1)H₃), 1.49-1.31 (m, 4H), 0.90 (t, J=7.2, C(7)H₃). ¹³C-NMR (100 MHz, CDCl₃): δ 200.26 (s, C(2)), 143.08 (s, C(3)), 139.34 (d, CH═C(3)), 135.84 (s), 129.20 (d, 2 C), 128.51 (d, 2 C), 128.47 (d), 31.32 (t), 26.15 (q, C(1)), 26.13 (t), 22.96 (t), 13.82 (q, C(7)). MS (EI): 203 (6), 202 (41), 201 (35), 187 (20), 173 (5), 159 (35), 145 (16), 131 (16), 129 (53), 117 (72), 115 (57), 91 (52), 43 (100).

### EXAMPLE 10

### (E)-3-benzylideneoctan-2-one (compound ID 5) and (1E,4E)-4-benzylidene-1-phenylnon-1-en-3-one

As described in Example 6, the reaction of benzaldehyde and diethyl 2-oxooctan-3-ylphosphonate (obtained from hexyl iodide and triethyl phosphite via diethyl hexylphosphonate) in 1:2 water/dichloromethane gave after FC, (E)-3-benzylideneoctan-2-one (22%) and (1E,4E)-4-benzylidene-1-phenylnon-1-en-3-one (30%).

(E)-3-benzylideneoctan-2-one (Boiling point: 80° C. (0.08 mbar):

¹H-NMR (400 MHz, CDCl₃): δ 7.47 (s, H—C═C(3)), 7.44-7.32 (m, 5H), 2.51-2.46 (m, 2H—C(4)), 2.45 (s, C(1)H₃), 1.50-1.40 (m, 2H), 1.37-1.25 (m, 4H), 0.88 (t, J=7.1, C(8)H₃). MS (EI): 217 (3), 216 (19), 201 (8), 173 (3), 159 (15), 145 (8), 129 (30), 117 (28), 115 (25), 91 (30), 43 (100).

(1E,4E)-4-benzylidene-1-phenylnon-1-en-3-one (Boiling point 180° C. at 0.07 mbar):

¹³C-NMR (100 MHz, CDCl₃): δ 193.15 (s, C(3)), 143.90 (s), 143.52 (d), 138.03 (d), 135.93 (s), 135.11 (s), 130.21 (d), 129.24 (d, 2 C), 128.90 (d, 2 C), 128.54 (d, 2 C), 128.41 (d), 128.26 (d, 2 C), 122.79 (d), 32.04 (t), 28.69 (t), 27.22 (t), 22.41 (t), 14.03 (q, C(9)).

### EXAMPLE 11

### (E)-3-benzylidenenonan-2-one (Compound ID 6)

Prepared as described in Example 6 in 16% yield from benzaldehyde and diethyl 2-oxononan-3-ylphosphonate (obtained from heptyl iodide and triethyl phosphite via diethyl heptylphosphonate). Boiling point: 108° C. (0.08 mbar).

¹³C-NMR (100 MHz, CDCl₃): δ 200.26 (s, C(2)), 143.09 (s, C(3)), 139.35 (d, CH═C(3)), 135.84 (s), 129.21 (d, 2 C), 128.52 (d, 2 C), 128.47 (d), 31.52 (t), 29.52 (t), 29.11 (t), 26.38 (t), 26.16 (q, C(1)), 22.58 (t), 14.05 (q, C(9)). MS (EI): 231 (5), 230 (27), 229 (20), 215 (11), 187 (4), 173 (4), 159 (32), 145 (19), 129 (71), 117 (57), 115 (46), 91 (61), 43 (100).

### EXAMPLE 12

### 3-phenylmethylheptan-2-one

In an autoclave, a solution of (E)-3-benzylideneheptan-2-one (350 mg, 1.7 mmol, prepared as described in Example 9) in ethanol (5 ml) was stirred for 17 h under hydrogen (12 bars) in the presence of Pd/C (10%, 40 mg). The mixture was filtered over Celite and the solvent evaporated to give 3-phenylmethylheptan-2-one (350 mg, 99%). Boiling point: 65° C. (0.11 mbar).

¹H-NMR (400 MHz, CDCl₃): δ 7.31-7.11 (m, 5H), 2.87 (dd, J=8.2, 12.6, 1H), 2.86-2.76 (m, 1H), 2.68 (dd, J=5.4, 12.8, 1H), 1.99 (s, C(1)H₃), 1.69-1.58 (m, 1H), 1.51-1.40 (m, 1H), 1.35-1.18 (m, 4H), 0.87 (t, J=6.9, C(7)H₃). MS (EI): 204 (2), 189 (2), 148 (26), 147 (73), 131 (1), 129 (7), 105 (11), 91 (100), 65 (11), 43 (32). IR: νₘₐₓ 3028, 3007, 2930, 2859, 1712, 1603, 1497, 1455, 1351, 1215, 1162, 1115, 1079, 1030, 946, 917, 741, 699 cm⁻¹.

### EXAMPLE 13

### 3-phenylmethyloctan-2-one (Compound ID 4)

Prepared in 75% yield as described in Example 12 by hydrogenation of (E)-3-benzylideneoctan-2-one (400 mg, 1.8 mmol, prepared as described in Example 10). Boiling point: 70° C. (0.09 mbar).

¹H-NMR (400 MHz, CDCl₃): δ 7.30-7.11 (m, 5H), 2.87 (dd, J=8.3, 12.6, 1H), 2.85-2.77 (m, 1H), 2.68 (dd, J=5.4, 12.5, 1H), 1.99 (s, C(1)H₃), 1.68-1.57 (m, 1H), 1.50-1.39 (m, 1H), 1.33-1.19 (m, 6H), 0.87 (t, J=6.8, C(8)H₃). MS (EI): 218 (2), 203 (2), 149 (3), 148 (34), 147 (86), 129 (7), 117 (11), 115 (7), 105 (12), 91 (100), 65 (10), 43 (35). IR: νₘₐₓ 3064, 3028, 3007, 2929, 2858, 1712, 1603, 1496, 1455, 1352, 1162, 121, 1079, 1030, 950, 752, 700 cm⁻¹.

### EXAMPLE 14

### (E)-4-(2-acetylhept-1-enyl)benzonitrile (Compound ID 7)

Prepared as described in Example 6 in 10% yield from 4-cyanobenzaldehyde and diethyl 2-oxooctan-3-ylphosphonate (obtained from hexyl iodide and triethyl phosphite via diethyl hexylphosphonate). Boiling point: 205° C. (0.07 mbar).

¹³C-NMR (100 MHz, CDCl₃): δ 199.73 (s), 145.44 (s, C(2)), 140.55 (s), 136.56 (d, C(1)), 132.25 (d, 2 C), 129.56 (d, 2 C), 118.50 (s, CN), 111.84 (s), 31.93 (t), 28.81 (t), 26.49 (t), 26.26 (q, C(1)), 22.30 (t), 13.94 (q, C(7)). MS (EI): 241 (14), 226 (13), 212 (8), 198 (8), 184 (21), 170 (23), 156 (31), 154 (34), 142 (53), 130 (12), 116 (30), 43 (100).

### EXAMPLE 15

### (E)-3-(naphthalen-2-ylmethylene)octan-2-one (Compound ID 8)

Prepared as described in Example 6 in 3% yield from 2-naphtaldehyde and diethyl 2-oxooctan-3-ylphosphonate (obtained from hexyl iodide and triethyl phosphite via diethyl hexylphosphonate). Boiling point: 220° C. (0.07 mbar).

¹³C-NMR (100 MHz, CDCl₃): δ 200.26 (s, C(2)), 143.31 (s, C(3)), 139.41 (d, CH═C(3)), 133.33 (s), 133.16 (s), 133.01 (s), 129.01 (d), 128.32 (d), 128.13 (d), 127.65 (d), 126.78 (d), 126.66 (d), 126.51 (d), 32.12 (t), 28.92 (t), 26.48 (t), 26.22 (q, C(1)), 22.40 (t), 14.05 (q, C(8)). MS (EI): 267 (13), 266 (64), 265 (35), 251 (7), 223 (12), 209 (35), 195 (18), 179 (73), 167 (70), 165 (79), 152 (36), 141 (65), 128 (62), 115 (15), 43 (100).

### EXAMPLE 16

### (E)-3-(thiophen-2-ylmethylene)octan-2-one (Compound ID 9)

Prepared as described in Example 6 in 22% yield from 2-thiophencarboxaldehyde and diethyl 2-oxooctan-3-ylphosphonate (obtained from hexyl iodide and triethyl phosphite via diethyl hexylphosphonate). Boiling point: 115° C. (0.08 mbar).

¹H-NMR (400 MHz, CDCl₃): δ 7.62 (s, H—C═C(3)), 7.52 (dt, J=0.9, 5.2, 1H), 7.29 (ddd, J=0.6, 1.1, 3.6, 1H), 7.12 (dd, J=3.8, 7.1, 1H), 2.68-2.62 (m, 2H—C(4)), 2.43 (s, C(1)H₃), 1.50-1.31 (m, 6H), 0.91 (t, J=7.2, C(8)H₃). MS (EI): 222 (20), 207 (7), 179 (16), 165 (13), 151 (9), 137 (14), 135 (12), 123 (42), 109 (15), 97 (31), 43 (100). IR: νₘₐₓ 2955, 2927, 2859, 1657, 1609, 1456, 1420, 1389, 1356, 1259, 1204, 1124, 1053, 968, 943, 885, 857, 702, 634 cm⁻¹.

### EXAMPLE 17

### (E)-3-(furan-2-ylmethylene)octan-2-one

Prepared as described in Example 6 in 55% yield from 2-furancarboxaldehyde and diethyl 2-oxooctan-3-ylphosphonate (obtained from hexyl iodide and triethyl phosphite via diethyl hexylphosphonate). Boiling point: 95° C. (0.09 mbar).

¹H-NMR (400 MHz, CDCl₃): δ 7.56 (dd, J=0.6, 1.8, 1H), 7.21 (s, H—C═C(3)), 6.66 (d, J=3.5, 1H), 6.53 (dd, J=1.8, 3.5, 1H), 2.68-2.62 (m, 2H—C(4)), 2.40 (s, C(1)H₃), 1.47-1.29 (m, 6H), 0.90 (t, J=7.1, C(8)H₃). MS (EI): 206 (38), 191 (10), 177 (3), 163 (23), 150 (11), 149 (20), 135 (11), 121 (20), 107 (47), 95 (8), 91 (14), 81 (25), 43 (100). IR: νₘₐₓ 2956, 2929, 2860, 1660, 1622, 1547, 1475, 1377, 1349, 1279, 1255, 1206, 1151, 1123, 1090, 1020, 983, 928, 884, 742 cm⁻¹.

### EXAMPLE 18

### 3-((tetrahydrofuran-2-yl)methyl)octan-2-one

Prepared as described in Example 17 by hydrogenation of (E)-3-(furan-2-ylmethylene)octan-2-one (prepared as described in Example 12) in 30% yield and as a 53:47 mixture of diastereomers. Boiling point: 80° C. (0.13 mbar).

¹H-NMR (400 MHz, CDCl₃): δ 3.86-3.74 (m, 3H), 3.74-3.63 (m, 3H), 2.79-2.71 (m, 1H), 2.64-2.56 (m, 1H), 2.17 (s, C(1)H₃), 2.16 (s, C(1)H₃), 2.03-1.77 (m, 8H), 1.64-1.35 (m, 8H), 1.34-1.17 (m, 12H), 0.87 (t, J=6.9, 2 C(8)H₃). MS (EI): major diast 212 (1), 142 (5), 128 (2), 95 (6), 85 (93), 71 (100), 67 (9), 55 (14), 43 (65). MS (EI): minor diast 212 (1), 142 (11), 128 (2), 95 (8), 85 (54), 71 (100), 67 (10), 55 (14), 43 (59). IR: νₘₐₓ 2956, 2929, 2858, 1712, 1461, 1354, 1165, 1066, 952, 881, 722 cm⁻¹.

### EXAMPLE 19

### (E)-3-((tetrahydrofuran-3-yl)methylene)heptan-2-one (Compound ID 10)

Prepared as described in Example 6 in 30% yield from tetrahydro-3-furancarboxaldehyde and diethyl 2-oxoheptan-3-ylphosphonate (obtained from pentyl iodide and triethyl phosphite via diethyl pentylphosphonate). Boiling point: 75° C. (0.08 mbar).

¹H-NMR (400 MHz, CDCl₃): δ 6.46 (d, J=9.6, H—C═C(3)), 4.02-3.94 (m, 2H), 3.85 (dt, J=7.4, 8.1, 1H), 3.51 (dd, J=7.1, 8.6, 1H), 3.28-3.17 (m, 1H), 2.33-2.28 (m, 2H), 2.30 (s, C(1)H₃), 2.27-2.16 (m, 1H), 1.76 (dq, J=7.8, 12.4, 1H), 1.38-1.22 (m, 4H), 0.90 (t, J=6.8, C(7)H₃). MS (EI): 196 (9), 181 (3), 165 (12), 151 (61), 138 (5), 125 (8), 123 (10), 109 (17), 95 (26), 81 (24), 67 (15), 55 (15), 43 (100). IR: νₘₐₓ 2956, 2929, 2861, 1667, 1638, 1453, 1384, 1351, 1261, 1202, 1146, 1123, 1068, 956, 910, 723 cm⁻¹.

### EXAMPLE 20

### (E)-3-((tetrahydrofuran-3-yl)methylene)octan-2-one

Prepared as described in Example 6 in 20% yield from tetrahydro-3-furancarboxaldehyde and diethyl 2-oxooctan-3-ylphosphonate (obtained from hexyl iodide and triethyl phosphite via diethyl hexylphosphonate). Boiling point: 80° C. at 0.08 mbar.

¹H-NMR (400 MHz, CDCl₃): δ 6.46 (d, J=9.9, H—C═C(3)), 4.02-3.94 (m, 2H), 3.85 (dt, J=7.4, 8.1, 1H), 3.51 (dd, J=7.1, 8.6, 1H), 3.28-3.17 (m, 1H), 2.33-2.27 (m, 2H), 2.30 (s, C(1)H₃), 2.27-2.16 (m, 1H), 1.76 (dq, J=7.8, 12.4, 1H), 1.38-1.22 (m, 6H), 0.88 (t, J=6.9, C(8)H₃). MS (EI): 210 (11), 209 (4), 195 (3), 179 (12), 165 (66), 153 (5), 139 (11), 123 (11), 109 (19), 95 (28), 81 (20), 67 (13), 55 (15), 43 (100).

### EXAMPLE 21

### 3-((tetrahydrofuran-3-yl)methyl)octan-2-one

Prepared as described in Example 12 by hydrogenation of (E)-3-((tetrahydrofuran-3-yl)methylene)octan-2-one (prepared as described in Example 20) in 85% yield and as a 1:1 mixture of diastereomers. Boiling point: 80° C. (0.08 mbar).

¹H-NMR (400 MHz, CDCl₃): δ 3.92-3.81 (m, 2H), 3.76-3.69 (m, 1H), 3.30 (ddd, J=7.3, 8.4, 15.7, 1H), 2.52-2.39 (m, 1H), 2.14 (s, C(1)H₃), 2.16-1.97 (m, 2H), 1.79-1.69 (m, 1H), 1.64-1.53 (m, 1H), 1.53-1.36 (m, 3H), 1.35-1.18 (m, 6H), 0.88 (t, J=6.8, C(8)H₃). MS (EI): (1:1 mixture) 213 (3), 212 (19), 194 (3), 155 (40), 142 (31), 128 (20), 109 (19), 95 (37), 85 (23), 83 (61), 81 (27), 71 (59), 69 (32), 55 (62), 43 (100). IR: νₘₐₓ 2956, 2929, 2857, 1711, 1454, 1353, 1165, 1049, 963, 906, 722, 666 cm⁻¹.

### EXAMPLE 22

### (E)-3-(2,2-dimethoxyethylidene)heptan-2-one

Prepared as described in Example 6 in 28% yield from dimethoxyacetaldehyde and diethyl 2-oxoheptan-3-ylphosphonate (obtained from pentyl iodide and triethyl phosphite via diethyl pentylphosphonate). Boiling point: 50° C. (0.09 mbar).

¹H-NMR (400 MHz, CDCl₃): δ 6.41 (d, J=6.4, H—C═C(3)), 5.15 (d, J=6.3, H—C(OMe)₂), 3.37 (s, 2 MeO), 2.33 (s, C(1)H₃), 2.37-2.30 (m, 2H), 1.37-1.27 (m, 4H), 4H), 0.90 (t, J=7.1, C(7)H₃). MS (EI): 200 (1), 185 (1), 169 (29), 168 (14), 157 (45), 137 (5), 125 (20), 111 (24), 95 (34), 75 (56), 55 (21), 43 (100). IR: νₘₐₓ 2957, 2927, 2830, 1678, 1457, 1355, 1254, 1192, 1132, 1089, 1054, 975, 914, 725 cm⁻¹.

### EXAMPLE 23

### (E)-3-(2,2-dimethoxyethylidene)octan-2-one (Compound ID 11)

Prepared as described in Example 6 in 30% yield from dimethoxyacetaldehyde and diethyl 2-oxooctan-3-ylphosphonate (obtained from hexyl iodide and triethyl phosphite via diethyl hexylphosphonate). Boiling point: 60° C. (0.09 mbar).

¹H-NMR (400 MHz, CDCl₃): δ 6.41 (d, J=6.3, H—C═C(3)), 5.15 (d, J=6.3, H—C(OMe)₂), 3.37 (s, 2 MeO), 2.33 (s, C(1)H₃), 2.35-2.29 (m, 2H), 1.39-1.24 (m, 6H), 0.88 (t, J=6.9, C(8)H₃). MS (EI): 214 (1), 183 (30), 171 (36), 157 (23), 139 (13), 125 (11), 111 (23), 95 (18), 75 (69), 55 (22), 43 (100). IR: νₘₐₓ 2957, 2931, 2830, 1678, 1459, 1355, 1248, 1192, 1132, 1091, 1054, 963, 915, 723 cm⁻¹.

### EXAMPLE 24

### 3-(2,2-dimethoxyethyl)octan-2-one and 3-(2-methoxyethyl)octan-2-one

Hydrogenation (as described in Example 12) of (E)-3-(2,2-dimethoxyethylidene)octan-2-one (prepared as described in Example 23) gave a 2:1 mixture of 3-(2,2-dimethoxyethyl)octan-2-one and of 3-(2-methoxyethyl)octan-2-one. Flash chromatography (FC) (70 g SiO₂, hexane/diethyl ether 6:1) of the crude product (0.67 g) gave 3-(2-methoxyethyl)octan-2-one (0.12 g, 20%) and 3-(2,2-dimethoxyethyl)octan-2-one (0.24 g, 35%).

3-(2-Methoxyethyl)octan-2-one (Boiling point: 45° C. at 0.09 mbar):

¹H-NMR (400 MHz, CDCl₃): δ 3.36-3.30 (m, CH₂OMe), 3.29 (s, MeO), 2.65-2.57 (m, 1H), 2.15 (s, C(1)H₃), 1.95-1.84 (m, 1H), 1.70-1.53 (m, 2H), 1.46-1.35 (m, 1H), 1.33-1.20 (m, 6H), 0.87 (t, J=7.0, C(8)H₃). MS (EI): 186 (1), 154 (2), 139 (1), 128 (47), 116 (25), 97 (17), 84 (37), 71 (100), 69 (40), 55 (31), 45 (50), 43 (64). IR: νₘₐₓ 2956, 2928, 2859, 1712, 1459, 1352, 1167, 1118, 959 cm⁻¹.

3-(2,2-Dimethoxyethyl)octan-2-one (Boiling point: 70° C. at 0.09 mbar):

¹H-NMR (400 MHz, CDCl₃): δ 4.30 (t, J=5.6, H—C(OMe)₂), 3.30 (s, MeO), 3.29 (s, MeO), 2.66-2.56 (m, 1H), 2.15 (s, C(1)H₃), 2.00 (ddd, J=5.7, 9.7, 14.1, 1H), 1.65 (ddd, J=4.3, 5.7, 14.1, 1H), 1.62-1.51 (m, 1H), 1.44-1.34 (m, 1H), 1.33-1.20 (m, 6H), 0.87 (t, J=6.9, C(8)H₃). MS (EI): 215 (1), 185 (15), 153 (6), 141 (6), 127 (18), 114 (21), 95 (13), 89 (12), 88 (7), 75 (100), 71 (34), 67 (7), 43 (21). IR: νₘₐₓ 2956, 2930, 2859, 1713, 1458, 1362, 1192, 1167, 1123, 1060, 947 cm⁻¹.

### EXAMPLE 25

### (E)-3-(2-(2-methyl-1,3-dioxolan-2-yl)ethylidene)octan-2-one (Compound ID 12)

Prepared as described in Example 6 in 24% yield from 2-(2-methyl-1,3-dioxolan-2-yl)acetaldehyde (prepared from ethyl acetoacetate by acetalisation with ethylene glycol in toluene in the presence of p-toluenesulfonic acid monohydrate followed by reduction using diisobutylaluminium hydride (1 M solution in hexane) in 10:1 hexane/tetrahydrofuran) and diethyl 2-oxooctan-3-ylphosphonate (obtained from hexyl iodide and triethyl phosphite via diethyl hexylphosphonate). Boiling point: 90° C. (0.09 mbar).

¹H-NMR (400 MHz, CDCl₃): δ 6.64 (t, J=7.2, H—C═C(3)), 4.03-3.96 (m, (OCH₂)₂), 2.61 (d, J=7.1, CH₂CH═), 2.32 (s, C(1)H₃), 2.30-2.24 (m, 2H), 1.36 (s, Me), 1.34-1.24 (m, 6H), 0.87 (t, J=6.9, C(8)H₃). MS (EI): 225 (1), 87 (100), 53 (3), 43 (44). IR: νₘₐₓ 2956, 2930, 2873, 1668, 1455, 1378, 1351, 1213, 1114, 1079, 1046, 948, 857, 784 cm⁻¹.

### EXAMPLE 26

### 3-(2-(2-methyl-1,3-dioxolan-2-yl)ethyl)octan-2-one

Prepared as described in Example 12 by hydrogenation of (E)-3-(2-(2-methyl-1,3-dioxolan-2-yl)ethylidene)octan-2-one (prepared as described in Example 25) in 69% is yield. Boiling point: 95° C. (0.08 mbar).

¹H-NMR (400 MHz, CDCl₃): δ 3.97-3.87 (m, (OCH₂)₂), 2.48-2.39 (m, 1H), 2.12 (s, C(1)H₃), 1.74-1.48 (m, 5H), 1.46-1.35 (m, 1H), 1.30 (s, Me), 1.30-1.19 (m, 6H), 0.87 (t, J=6.9, C(8)H₃). MS (EI): 242 (1), 227 (2), 172 (1), 99 (15), 87 (100), 71 (4), 55 (8), 43 (39). IR: νₘₐₓ 2956, 2930, 2873, 1710, 1457, 1376, 1353, 1216, 1169, 1143, 1053, 948, 862, 786, 717 cm⁻¹.

### EXAMPLE 27

### 3-pentylheptane-2,6-dione

A solution of 3-(2-(2-methyl-1,3-dioxolan-2-yl)ethyl)octan-2-one (0.5 g, 2 mmol, prepared as described in Example 26) in tetrahydrofuran (20 ml) was treated with water (0.05 ml) and concentrated HCl (0.075 ml) and stirred at 20° C. for 8 h. The resulting mixture was poured into water and extracted with diethyl ether. The organic phases were washed with saturated aqueous NaHCO₃ solution and dried (Na₂SO₄). Flash chromatography (FC) (50 g SiO₂, hexane/diethyl ether 3:2) of the crude product (0.45 g) gave 3-pentylheptane-2,6-dione (0.34 g, 83%). Boiling point: 70° C. (0.09 mbar).

¹H-NMR (400 MHz, CDCl₃): δ 2.50-2.27 (m, 3H), 2.13 (s, C(1)H₃, C(7)H₃), 1.89-1.55 (m, 3H), 1.45-1.34 (m, 1H), 1.34-1.19 (m, 6H), 0.88 (t, J=6.9, Me). MS (EI): 198 (1), 141 (7), 128 (24), 110 (8), 100 (9), 95 (14), 85 (12), 71 (21), 58 (21), 55 (17), 43 (100).

### EXAMPLE 28

### (E)-3-ethylideneoctan-2-one

A) A mixture of diethyl 2-oxooctan-3-ylphosphonate (2.0 g, 7.6 mmol) and LiOH.H₂O (0.32 g, 7.6 mmol) in tetrahydrofuran (30 ml) was stirred for 35 min. at 20° C. A solution of acetaldehyde (0.37 g, 8.3 mmol) in tetrahydrofuran (20 ml) was then added. The resulting suspension was stirred for 44 h and poured into saturated aqueous NH₄Cl. The aqueous phase was extracted with diethyl ether. The combined organic phases were washed with water (100 ml), dried (Na₂SO₄), and the solvent evaporated. Ball-to-ball distillation of the crude product (1.4 g) gave (E)-3-ethylideneoctan-2-one (0.36 g, 31%).

B) Prepared as described in Example 6 in 40% yield from acetaldehyde and diethyl 2-oxooctan-3-ylphosphonate (obtained from hexyl iodide and triethyl phosphite via diethyl hexylphosphonate). Boiling point: 65° C. (0.4 mbar).

¹³C-NMR (100 MHz, CDCl₃): δ 199.27 (s, C(2)), 143.45 (s, C(3)), 138.12 (d, CH═C(3)), 31.77 (t), 28.51 (t), 25.46 (q, C(1)), 24.88 (t), 22.38 (t), 13.86 (q, C(8)), 13.82 (q). MS (EI): 154 (8), 139 (49), 125 (15), 111 (11), 107 (4), 97 (15), 83 (26), 69 (60), 55 (57), 43 (100). IR: νₘₐₓ 2956, 2928, 2859, 1668, 1639, 1457, 1390, 1349, 1280, 1258, 1195, 1136, 1109, 1020, 959, 823, 721 cm⁻¹.

### EXAMPLE 29

### 1-(2-methyl-1-pentylcyclopropyl)ethanone

A mixture of NaH (0.21 g, 8.6 mmol) and Me₃SOI (1.89 g, 8.6 mmol) in DMSO (10 ml) was stirred for 30 min. and treated with (E)-3-ethylideneoctan-2-one (1.2 g, 7.8 mmol, prepared as described in Example 28). The resulting mixture was stirred for 1 h at 20° C. and 3 h at 60° C., cooled, and poured into saturated aqueous NaHCO₃. The aqueous phase was extracted with diethyl ether. The combined organic phases were washed with water (100 ml), dried (Na₂SO₄), and the solvent evaporated. Flash chromatography (FC) (200 g SiO₂, hexane/diethyl ether 95:5) of the crude product (1.43 g) gave 1-(2-methyl-1-pentylcyclopropyl)ethanone (0.9 g, 69%). Boiling point: 100° C. (10 mbar).

¹H-NMR (400 MHz, CDCl₃): δ 2.06 (s, C(2)H₃), 1.82-1.72 (m, 1H), 1.50-1.24 (m, 9H), 1.15 (d, J=6.1, Me), 0.89 (t, J=6.9, Me), 0.39 (dd, J=3.8, 6.1, 1H). MS (EI): 168 (1), 153 (1), 139 (2), 111 (100), 83 (6), 69 (13), 55 (17), 43 (57). IR: νₘₐₓ 2956, 2931, 2872, 1686, 1466, 1395, 1354, 1295, 1254, 1148, 1098, 1027, 964, 890, 826, 725 cm⁻¹.

### EXAMPLE 30

### 3-(propan-2-ylidene)octan-2-one

At −78° C., ammonia (250 ml) was treated with FeCl₃ (30 mg) and sodium (3.45 g, 0.15 mol). The dark blue mixture was shortly refluxed and the resulting dark grey mixture was treated dropwise with a solution of mesityl oxide (15 g, 0.15 mol) in diethyl ether (25 ml), stirred for 1 h and treated dropwise with a solution of pentyl iodide (30.76 g, 0.155 mol) in diethyl ether (10 ml). The resulting mixture was stirred for 1 h, treated with diethyl ether (100 ml) and the ammonia was evaporated by warming to 20° C. The residue was treated with water (50 ml), acidified by addition of 2N HCl, and extracted with diethyl ether. The combined organic phases were washed, dried (Na₂SO₄), and the solvent evaporated. Distillation of the crude product (27.2 g) at 120° C. and 50 mbar gave a fraction that was treated with PTSA.H₂O (200 mg) and refluxed for 1 h. Flash chromatography (FC) (700 g SiO₂, hexane/diethyl ether 95:8) of the resulting mixture gave 3-(propan-2-ylidene)octan-2-one (3.6 g, 14%). Boiling point: 45° C. (0.2 mbar).

¹H-NMR (400 MHz, CDCl₃): δ 2.28-2.22 (m, 2H), 2.24 (s, C(1)H₃), 1.80 (s, Me), 1.75 (s, Me), 1.40-1.22 (m, 6H), 0.88 (t, J=6.9, Me). MS (EI): 168 (11), 153 (69), 139 (2), 135 (3), 125 (6), 111 (29), 97 (11), 83 (28), 69 (83), 55 (25), 43 (100). IR: νₘₐₓ 2957, 2926, 2859, 1687, 1457, 1374, 1351, 1287, 1186, 1139, 1109, 968, cm⁻¹.

### EXAMPLE 31

### methyl 1-pentylcyclopentanecarboxylate (Compound ID 14)

A) At −70° C., a solution of diisopropylamine (45.9 ml, 0.33 mol) in tetrahydrofuran (200 ml) was treated within 1 h with a solution of 1.6 M n-butyllithium in tetrahydrofuran (200 ml, 0.33 mol). The resulting solution was warmed to 0° C. and treated within 10 min. with a solution of cyclopentanecarboxylic acid (14.3 ml, 0.13 mol) in tetrahydrofuran (25 ml). After 35 min. stirring at 4° C., pentyl iodide (39 g, 0.197 mol) was added and the solution was stirred for 19 h at 20° C. and then poured into 2M aqueous HCl (350 ml). The aqueous phase was extracted twice with methyl t-butyl ether (150 ml). The combined organic phases were washed three times with water (250 ml), dried (MgSO₄), and the solvent evaporated. Flash chromatography (FC) (700 g SiO₂, hexane/methyl t-butyl ether 2:1) of the crude product (33 g) gave 1-pentylcyclopentanecarboxylic acid (13.2 g, 55%).

B) A solution of 1-pentylcyclopentanecarboxylic acid (3 g, 16 mmol) in hexane (10 ml) was treated with phosphorus tribromide (0.61 ml, 6.5 mmol) and stirred 6 h at 20° C. The supernatant was added dropwise to a solution of methanol (0.79 ml, 19.5 mmol) and pyridine (2.62 ml, 33 mmol) in hexane (40 ml). The resulting mixture was stirred at 20° C. for 17 h, at 50° C. for 2.5 h, at reflux for 1.5 h, and then poured into 2M aqueous HCl (100 ml). The aqueous phase was extracted twice with methyl t-butyl ether (80 ml). The combined organic phases were washed twice with an aqueous sodium chloride solution (100 ml), dried (MgSO₄), and the solvent evaporated. Flash chromatography (FC) (SiO₂, hexane/methyl t-butyl ether 15:1) of the crude product (2.6 g) gave methyl 1-pentylcyclopentanecarboxylate (0.7 g, 22%). Boiling point: 80° C. (0.1 mbar).

¹³C-NMR (100 MHz, CDCl₃): δ 178.49 (s), 54.15 (s, C(1)), 51.59 (q, MeO), 39.29 (t), 35.98 (t, 2 C), 32.23 (t), 25.63 (t), 24.88 (t, 2 C), 22.46 (t), 13.96 (q). MS (EI): 198 (1), 167 (1), 157 (15), 139 (23), 128 (100), 100 (13), 97 (32), 87 (18), 83 (86), 81 (20), 69 (41), 67 (49), 59 (11), 57 (19), 55 (49), 41 (44).

### EXAMPLE 32

### 1-(1-pentylcyclopentyl)ethanone (Compound ID 15)

At −20° C., a solution of 1-pentylcyclopentanecarboxylic acid (prepared as described in Example 31, 3 g, 16 mmol) in tetrahydrofuran (60 ml) was treated with a 1.6 M solution of methyllithium in tetrahydrofuran (25.4 ml, 41 mmol). The resulting solution was stirred for 4 h at −10° C. and treated slowly with water (25 ml), then with a 2M solution of aqueous HCl (30 ml), and extracted twice with methyl t-butyl ether (80 ml). The combined organic phases were washed twice with water (100 ml), dried (MgSO₄), and the solvent evaporated. Flash chromatography (FC) (SiO₂, hexane/methyl t-butyl ether 25:1) of the crude product (2.8 g) gave methyl 1-pentylcyclopentanecarboxylate (0.6 g, 20%). Boiling point: 70° C. (0.1 mbar).

¹³C-NMR (100 MHz, CDCl₃): δ 212.56 (s), 60.34 (s), 38.95 (t), 34.34 (t, 2 C), 32.36 (t), 25.40 (t), 25.33 (q), 24.96 (t, 2 C), 22.39 (t), 13.93 (q). MS (EI): 182 (1), 167 (1), 139 (6), 112 (41), 97 (39), 83 (100), 69 (46), 67 (28), 57 (21), 55 (51), 43 (56), 41 (35).

### EXAMPLE 34

### (E)-3-(cyclohexylmethylene)octan-2-one (Compound ID 16)

Prepared as described in Example 6 in 10% yield from cyclohexanecarbaldehyde and diethyl 2-oxooctan-3-ylphosphonate (obtained from hexyl iodide and triethyl phosphite via diethyl hexylphosphonate). Boiling point: 119° C. (0.07 mbar).

¹³C-NMR (100 MHz, CDCl₃): δ 200.14 (s, C(2)), 148.59 (d, CH═C(3)), 140.46 (s, C(3)), 38.08 (d), 32.38 (t, 2 C), 31.93 (t), 29.53 (t), 25.82 (t), 25.68 (q, C(1)), 25.59 (t), 25.53 (t, 2 C), 22.46 (t), 13.97 (q, C(8)). MS (EI): 222 (9), 207 (4), 165 (81), 147 (20), 139 (20), 121 (8), 109 (17), 107 (14), 105 (8), 95 (19), 81 (24), 67 (26), 55 (34), 43 (100).

### EXAMPLE 35

### (E)-3-(cyclohex-3-enylmethylene)octan-2-one (Compound ID 17)

At 20° C., a solution of K₂CO₃ (53.3 g, 0.38 mol) in water (80 ml) was treated dropwise within 5 min. with a mixture of diethyl 2-oxooctan-3-ylphosphonate (6.0 g, 22.7 mmol) and 2,3,6-tetrahydrobenzaldehyde (3.9 ml, 34.1 mmol). The resulting mixture was then treated with a solution of tetrabutylammonium hydrogen sulfate (1.3 g, 3.8 mmol) in water (10 ml), stirred for 72 h at 20° C., poured into ice/water (100 ml), and extracted twice with cyclohexane (100 ml). The combined organic phases were washed twice with aqueous 1N HCl (50 ml), water (50 ml), saturated aqueous NaCl solution (50 ml), dried (MgSO₄), and the solvent evaporated. Flash chromatography (FC) (400 g SiO₂, hexane/methyl t-butyl ether 50:1) of the crude product (7.2 g) gave (E)-3-(cyclohex-3-enylmethylene)octan-2-one (1.47 g, 29%). Boiling point: 123° C. (0.07 mbar).

¹³C-NMR (100 MHz, CDCl₃): δ 199.99 (s, C(2)), 147.54 (d, CH═C(3)), 141.15 (s, C(3)), 126.97 (d), 125.36 (d), 33.75 (d), 31.96 (t), 30.76 (t), 29.59 (t), 28.19 (t), 25.73 (q, C(1)), 25.64 (t), 24.20 (t), 22.48 (t), 13.98 (q, C(8)). MS (EI): 220 (13), 205 (3), 177 (5), 166 (6), 163 (31), 151 (7), 149 (6), 145 (10), 139 (18), 137 (12), 131 (8), 123 (22), 109 (29), 95 (20), 93 (18), 92 (16), 91 (21), 85 (12), 81 (22), 80 (28), 79 (29), 77 (16), 67 (25), 55 (18), 43 (100).

### EXAMPLE 36

### (E)-3-(cyclopentylmethylene)octan-2-one (Compound ID 18)

Prepared as described in Example 6 in 15% yield from cyclopentanecarbaldehyde and diethyl 2-oxooctan-3-ylphosphonate (obtained from hexyl iodide and triethyl phosphite via diethyl hexylphosphonate). Boiling point: 102° C. (0.08 mbar).

¹³C-NMR (100 MHz, CDCl₃): δ 199.79 (s, C(2)), 148.77 (d, CH═C(3)), 140.86 (s, C(3)), 39.67 (d), 33.55 (t, 2C), 31.96 (t), 29.56 (t), 25.68 (q, C(1)), 25.65 (t, 3 C), 22.52 (t), 13.99 (q, C(8)). MS (EI): 208 (12), 193 (7), 165 (6), 152 (12), 151 (100), 139 (21), 133 (9), 123 (4), 109 (18), 107 (8), 105 (8), 95 (31), 85 (15), 81 (20), 67 (25), 55 (24), 43 (88).

### EXAMPLE 37

### (E)-3-(cyclobutylmethylene)octan-2-one (Compound ID 19)

Prepared as described in Example 35 in 50% yield from cyclobutanecarbaldehyde (obtained by PCC-oxidation of cyclobutanemethanol) and diethyl 2-oxooctan-3-ylphosphonate (obtained from hexyl iodide and triethyl phosphite via diethyl hexylphosphonate). Boiling point: 105° C. (0.07 mbar).

¹³C-NMR (100 MHz, CDCl₃): δ 199.75 (s, C(2)), 147.99 (d, CH═C(3)), 140.66 (s, C(3)), 34.82 (d), 31.92 (t), 29.33 (t), 29.20 (t, 2 C), 25.66 (t), 25.61 (q, C(1)), 22.50 (t), 19.05 (t), 13.97 (q, C(8)). MS (EI): 194 (2), 179 (7), 166 (16), 151 (11), 137 (29), 123 (44), 109 (39), 95 (27), 81 (27), 79 (13), 77 (8), 67 (30), 55 (13), 43 (100).

### EXAMPLE 38

### (E)-3-(2-fluorobenzylidene)octan-2-one (Compound ID 20)

Prepared as described in Example 35 in 9% yield from 2-fluorobenzaldehyde and diethyl 2-oxooctan-3-ylphosphonate (obtained from hexyl iodide and triethyl phosphite via diethyl hexylphosphonate)._Boiling point: 130° C. (0.08 mbar).

¹³C-NMR (100 MHz, CDCl₃): δ 199.96 (s, C(2)), 160.24 (s (d), J=248.8), 144.85 (s, C(3)), 131.65 (d (d), J=3.3, CH═C(3)), 130.27 (d (d), J=8.3), 129.91 (d (d), J=2.5), 124.00 (d (d), J=3.3), 123.83 (s (d), J=14.1), 115.63 (d (d), J=22.4), 31.93 (t), 28.71 (t), 26.62 (t), 26.23 (q, C(1)), 22.30 (t), 13.94 (q, C(8)). MS (EI): 234 (18), 219 (13), 215 (2), 205 (6), 191 (4), 177 (11), 163 (12), 149 (19), 147 (21), 135 (43), 132 (1), 109 (50), 43 (100).

### EXAMPLE 39

### (E)-3-(3-fluorobenzylidene)octan-2-one (Compound ID 21)

Prepared as described in Example 35 in 40% yield from 3-fluorobenzaldehyde and diethyl 2-oxooctan-3-ylphosphonate (obtained from hexyl iodide and triethyl phosphite via diethyl hexylphosphonate). Boiling point: 126° C. (0.07 mbar).

¹³C-NMR (100 MHz, CDCl₃): δ 200.02 (s, C(2)), 162.73 (s (d), J=246.3), 144.05 (s, C(3)), 138.01 (s (d), J=7.4), 137.69 (d (d), J=2.0, CH═C(3)), 130.05 (d (d), J=8.3), 124.95 (d (d), J=2.5), 115.82 (d (d), J=22.4), 115.32 (d (d), J=20.7), 31.96 (t), 28.76 (t), 26.34 (t), 26.17 (q, C(1)), 22.32 (t), 13.95 (q, C(8)). MS (EI): 234 (44), 219 (16), 215 (1), 205 (6), 201 (1), 191 (7), 177 (24), 163 (17), 149 (23), 147 (46), 135 (54), 133 (37), 115 (11), 109 (51), 43 (100).

### EXAMPLE 40

### (E)-3-(4-fluorobenzylidene)octan-2-one (Compound ID 22)

Prepared as described in Example 6 in 41% yield from 4-fluorobenzaldehyde and diethyl 2-oxooctan-3-ylphosphonate (obtained from hexyl iodide and triethyl phosphite via diethyl hexylphosphonate). Boiling point: 120° C. (0.06 mbar).

¹³C-NMR (100 MHz, CDCl₃): δ 200.04 (s, C(2)), 162.58 (s (d), J=249.6), 142.93 (s (d), J=1.0, C(3)), 138.08 (d (d), J=2.0, CH═C(3)), 131.90 (s (d), J=3.3), 130.06 (d (d), J=8.3, 2 C), 115.60 (d (d), J=21.6, 2 C), 32.03 (t), 28.75 (t), 26.25 (t), 26.10 (q, C(1)), 22.36 (t), 13.97 (q, C(8)). MS (EI): 234 (11), 219 (14), 205 (2), 191 (4), 177 (15), 163 (8), 163 (8), 149 (13), 147 (23), 135 (30), 132 (1), 109 (41), 43 (100).

### EXAMPLE 41

### (E)-3-(2,6-difluorobenzylidene)octan-2-one (Compound ID 23)

At 0° C., a solution of diethyl 2-oxooctan-3-ylphosphonate (6 g, 22.7 mmol, obtained from hexyl iodide and triethyl phosphite via diethyl hexylphosphonate) and DBU (5.2 ml, 34.1 mmol) in dichloromethane (30 ml) was treated dropwise with a solution of 2,6-difluorobenzaldehyde (4.94 g, 34.1 mmol) in dichloromethane (20 ml). The resulting solution was stirred for 6.5 h at 0° C. and for 36 h at −15° C., poured into ice-cold water and extracted three times with hexane (100 ml). The combined organic phases were washed four times with a saturated aqueous NaCl solution, dried (MgSO₄), and the solvent evaporated. Ball-to-ball distillation (9 mbar, till 150° C.) of the crude product (7.5 g) followed by FC (280 g SiO₂, hexane/methyl t-butyl ether 50:1) of the residue (5.66 g) gave (E)-3-(2,6-difluorobenzylidene)octan-2-one (3.4 g, 60%). Boiling point: 105° C. (0.08 mbar).

¹³C-NMR (100 MHz, CDCl₃): δ 199.27 (s, C(2)), 159.71 (s (m), 2 C), 148.11 (s, C(3)), 129.89 (d (t), J_{C,F}=10.2), 125.60 (d), 113.32 (s (t), J_{C,F}=20.1), 111.38 (d (m), 2 C), 31.73 (t), 27.91 (t (t), J_{C,F}=1.7), 27.71 (t), 26.37 (q, C(1)), 22.18 (t), 13.82 (q, C(8)). MS (EI): 252 (18), 237 (19), 233 (3), 232 (2), 223 (14), 209 (5), 196 (3), 195 (6), 189 (8), 181 (15), 167 (20), 153 (46), 151 (22), 141 (9), 133 (12), 127 (64), 43 (100).

### EXAMPLE 42

### (E)-3-(2,4-difluorobenzylidene)octan-2-one (Compound ID 24)

Prepared as described in Example 41 in 37% yield from 2,4-difluorobenzaldehyde and diethyl 2-oxooctan-3-ylphosphonate (obtained from hexyl iodide and triethyl phosphite via diethyl hexylphosphonate). Boiling point: 100° C. (0.08 mbar).

¹³C-NMR (100 MHz, CDCl₃): δ 199.75 (s, C(2)), 163.00 (s (dd), J=11.6, 251.3), 160.43 (s (dd), J=11.6, 251.3), 144.86 (s, C(3)), 130.72 (d (dd), J=4.2, 9.6), 130.48 (d (br. d), J=3.6), 119.99 (s (dd), J=4.2, 13.7), 111.38 (d (dd), J=3.7, 21.6), 104.16 (d (t), J=25.5), 31.92 (t), 28.65 (t), 26.62 (t), 26.19 (q, C(1)), 22.29 (t), 13.92 (q, C(8)). MS (EI): 252 (13), 237 (21), 233 (2), 232 (1), 223 (7), 209 (5), 195 (9), 189 (3), 181 (12), 167 (18), 165 (14), 153 (43), 151 (25), 141 (5), 133 (10), 127 (62), 43 (100).

### EXAMPLE 43

### (Z)-3-(3,5-difluorobenzylidene)octan-2-one (Compound ID 26) and (E)-3-(3,5-difluorobenzylidene)octan-2-one (Compound ID 25)

Prepared as described in Example 41 in 39% yield from 3,5-difluorobenzaldehyde (4.0 g, 28.4 mmol) and diethyl 2-oxooctan-3-ylphosphonate (5.0 g, 18.9 mmol, obtained from hexyl iodide and triethyl phosphite via diethyl hexylphosphonate). FC (450 g SiO₂, hexane/methyl t-butyl ether 50:1) of crude product (5.57 g) gave (Z)-3-(3,5-difluorobenzylidene)octan-2-one (0.21 g, 4%) and (E)-3-(3,5-difluorobenzylidene)octan-2-one (1.85 g, 39%).

(Z)-3-(3,5-difluorobenzylidene)octan-2-one:

Boiling point: 110° C. (0.08 mbar).

¹H-NMR (400 MHz, CDCl₃): δ 6.76-6.68 (m, 3H), 6.45 (s, H—C═C(3)), 2.39-2.32 (m, 2H—C(4)), 2.09 (s, C(1)H₃), 1.53-1.42 (m, 2H), 1.40-1.27 (m, 4H), 0.91 (t, J=7.0, C(8)H₃). ¹³C-NMR (100 MHz, CDCl₃): δ 207.02 (s, C(2)), 162.91 (s (dd), J=13.1, 249.0, 2 C), 147.40 (s), 139.52 (s (t), J=9.5), 126.71 (d(br. t), J=2.7), 111.12 (d (m), 2 C), 103.13 (d (t), J=25.5), 35.33 (t), 31.34 (t), 30.64 (q, C(1)), 27.59 (t), 22.37 (t), 13.94 (q, C(8)).

(E)-3-(3,5-difluorobenzylidene)octan-2-one:

Boiling point: 105° C. (0.08 mbar).

¹H-NMR (400 MHz, CDCl₃): δ 7.32 (s, H—C═C(3)), 6.92-6.85 (m, 2H), 6.80 (tt, J=2.3, 8.8, 1H), 2.49-2.42 (m, 2H—C(4)), 2.43 (s, C(1)H₃), 1.47-1.37 (m, 2H), 1.36-1.27 (m, 4H), 0.88 (t, J=7.1, C(8)H₃). ¹³C-NMR (100 MHz, CDCl₃): δ 199.69 (s, C(2)), 162.93 (s (dd), J=12.9, 248.8, 2 C), 144.95 (s), 138.97 (s (t), J=9.5), 136.31 (d (br. t), J=2.5), 111.83 (d (m), 2 C), 103.69 (d (t), J=25.5), 31.89 (t), 28.69 (t), 26.37 (t), 26.16 (q, C(1)), 22.25 (t), 13.89 (q, C(8)). MS (EI): 252 (33), 237 (12), 233 (1), 223 (9), 209 (7), 195 (12), 189 (2), 181 (12), 167 (18), 165 (23), 153 (39), 151 (28), 141 (7), 133 (11), 127 (34), 43 (100).

### EXAMPLE 44

### (E)-3-(perfluorobenzylidene)octan-2-one (Compound ID 27)

Prepared as described in Example 41 in 13% yield from pentafluorobenzaldehyde and diethyl 2-oxooctan-3-ylphosphonate (obtained from hexyl iodide and triethyl phosphite via diethyl hexylphosphonate). Boiling point: 100° C. (0.09 mbar).

¹H-NMR (400 MHz, CDCl₃): δ 6.96 (br. s, H—C═C(3)), 2.46 (s, C(1)H₃), 2.22 (br. t, J=7.8, 2H—C(4)), 1.36-1.27 (m, 2H), 1.27-1.12 (m, 4H), 0.83 (t, J=7.0, C(8)H₃). MS (EI): 306 (14), 291 (8), 287 (1), 277 (9), 263 (10), 250 (3), 243 (11), 235 (10), 221 (9), 207 (15), 187 (15), 181 (21), 169 (3), 43 (100).

### EXAMPLE 45

### (E)-3-(2-methylbenzylidene)octan-2-one (Compound ID 28)

Prepared as described in Example 35 in 16% yield from 2-methylbenzaldehyde and diethyl 2-oxooctan-3-ylphosphonate (obtained from hexyl iodide and triethyl phosphite via diethyl hexylphosphonate). Boiling point: 110° C. (0.07 mbar).

¹³C-NMR (100 MHz, CDCl₃): δ 200.26 (s, C(2)), 143.58 (s, C(3)), 138.78 (d, CH═C(3)), 136.15 (s), 135.29 (s), 130.02 (d), 128.23 (d, 2 C), 125.68 (d), 31.84 (t), 28.85 (t), 26.33 (t), 26.26 (q, C(1)), 22.26 (t), 19.94 (q, MePh), 13.93 (q, C(8)). MS (EI): 230 (4), 229 (5), 216 (16), 215 (100), 197 (1), 187 (1), 173 (5), 159 (33), 145 (16), 143 (10), 131 (20), 129 (15), 128 (16), 117 (7), 116 (9), 115 (19), 105 (19), 91 (11), 43 (69).

### EXAMPLE 46

### (E)-3-(3-methylbenzylidene)octan-2-one (Compound ID 29)

Prepared as described in Example 35 in 26% yield from 3-methylbenzaldehyde and diethyl 2-oxooctan-3-ylphosphonate (obtained from hexyl iodide and triethyl phosphite via diethyl hexylphosphonate). Boiling point: 140° C. (0.09 mbar).

¹³C-NMR (100 MHz, CDCl₃): δ 200.27 (s, C(2)), 142.34 (s, C(3)), 139.49 (d, CH═C(3)), 138.70 (s), 132.93 (s), 129.35 (d, 2 C), 129.28 (d, 2 C), 32.10 (t), 28.79 (t), 26.36 (t), 26.11 (q, C(1)), 22.41 (t), 21.30 (q, MePh), 14.02 (q, C(8)). MS (EI): 230 (22), 229 (10), 216 (14), 215 (85), 197 (1), 187 (4), 173 (14), 159 (17), 145 (18), 143 (28), 131 (41), 129 (19), 128 (20), 116 (10), 115 (25), 105 (43), 91 (15), 43 (100).

### EXAMPLE 47

### (E)-3-(4-methylbenzylidene)octan-2-one (Compound ID 30)

Prepared as described in Example 35 in 28% yield from 4-methylbenzaldehyde and diethyl 2-oxooctan-3-ylphosphonate (obtained from hexyl iodide and triethyl phosphite via diethyl hexylphosphonate). Boiling point: 145° C. (0.09 mbar).

¹³C-NMR (100 MHz, CDCl₃): δ 200.28 (s, C(2)), 142.94 (s, C(3)), 139.57 (d, CH═C(3)), 138.10 (s), 135.77 (s), 130.01 (d), 129.27 (d), 128.41 (d), 126.25 (d), 32.05 (t), 28.82 (t), 26.37 (t), 26.13 (q, C(1)), 22.35 (t), 21.42 (q, MePh), 14.02 (q, C(8)). MS (EI): 230 (40), 229 (18), 216 (12), 215 (72), 197 (1), 187 (4), 173 (20), 159 (16), 145 (20), 143 (41), 131 (44), 129 (20), 128 (22), 116 (12), 115 (29), 105 (44), 91 (16), 43 (100).

### EXAMPLE 48

### (E)-3-(2-(trifluoromethyl)benzylidene)octan-2-one (Compound ID 31)

A mixture of 2-octanone (7.4 g, 56.3 mmol) and 2-(trifluoromethyl)benzaldehyde (5 g, 28 mmol) in acetic acid (35 ml) was treated dropwise with sulfuric acid (4.7 ml, 86 mmol). The resulting mixture was stirred at 40° C. for 6 h, cooled to 0° C., poured into ice/2N aqueous NaOH solution, and extracted three times with hexane (100 ml). The combined organic phases were washed three times with a saturated aqueous NaCl solution, dried (MgSO₄), and the solvent and the remaining starting materials evaporated. Ball-to-ball distillation (0.08 mbar) of the residue followed by FC (300 g SiO₂, hexane/methyl t-butyl ether 25:1) of the fraction distilling at 142° C. (3.17 g) gave (E)-3-(2-(trifluoromethyl)benzylidene)octan-2-one (1.57 g, 20%). Boiling point: 140° C. (0.08 mbar).

¹³C-NMR (100 MHz, CDCl₃): δ 199.91 (s, C(2)), 145.02 (s, C(3)), 135.73 (d, CH═C(3)), 134.70 (s (q), J=2), 131.59 (d), 129.98 (d), 128.37 (s (q), J=30), 128.04 (d), 126.01 (d (q), J=5), 121.28 (s (q), J=274, CF₃), 31.82 (t), 28.58 (t), 26.40 (t), 26.17 (q, C(1)), 22.19 (t), 13.81 (q, C(8)). MS (EI): 284 (19), 269 (5), 265 (1), 249 (4), 227 (9), 215 (100), 199 (11), 185 (23), 173 (16), 165 (21), 159 (37), 151 (10), 145 (9), 133 (9), 115 (11), 43 (88).

### EXAMPLE 49

### (E)-3-benzylidenehexan-2-one (Compound ID 32)

Prepared as described in Example 48 in 44% yield from benzaldehyde and 2-hexanone. Boiling point: 96° C. (0.08 mbar).

¹³C-NMR (100 MHz, CDCl₃): δ 200.26 (s, C(2)), 142.92 (s, C(3)), 139.50 (d, CH═C(3)), 135.83 (s), 129.21 (d, 2 C), 128.53 (d, 2 C), 128.48 (d), 28.37 (t), 26.13 (q, C(1)), 22.49 (t), 14.28 (q, C(6)). MS (EI): 188 (59), 187 (58), 173 (41), 159 (32), 145 (50), 129 (27), 117 (44), 115 (57), 105 (18), 91 (64), 43 (100).

### EXAMPLE 50

### (E)-3-(2-methoxybenzylidene)octan-2-one (Compound ID 34)

Prepared as described in Example 35 in 9% yield from 2-methoxybenzaldehyde and diethyl 2-oxooctan-3-ylphosphonate (obtained from hexyl iodide and triethyl phosphite via diethyl hexylphosphonate). Boiling point: 160° C. (0.08 mbar).

¹³C-NMR (100 MHz, CDCl₃): δ 200.46 (s, C(2)), 157.30 (s, C(3)), 142.81 (s), 135.45 (d, CH═C(3)), 129.95 (d), 129.56 (d), 124.92 (s), 120.28 (d), 110.49 (d), 55.47 (q, OMe), 32.01 (t), 28.95 (t), 26.55 (t), 26.27 (q, C(1)), 22.35 (t), 14.00 (q, C(8)). MS (EI): 246 (3), 231 (7), 215 (100), 203 (1), 189 (5), 175 (2), 161 (5), 159 (12), 147 (14), 131 (12), 121 (21), 115 (12), 108 (19), 91 (18), 43 (49).

### EXAMPLE 51

### (E)-3-(3-methoxybenzylidene)octan-2-one (Compound ID 35)

Prepared as described in Example 35 in 25% yield from 3-methoxybenzaldehyde and diethyl 2-oxooctan-3-ylphosphonate (obtained from hexyl iodide and triethyl phosphite via diethyl hexylphosphonate). Boiling point: 175° C. (0.09 mbar).

¹³C-NMR (100 MHz, CDCl₃): δ 200.26 (s, C(2)), 159.60 (s, C(3)), 143.27 (s), 139.22 (d, CH═C(3)), 137.17 (s), 129.51 (d), 121.68 (d), 114.43 (d), 114.22 (d), 55.22 (q, OMe), 32.11 (t), 28.92 (t), 26.48 (t), 26.17 (q, C(1)), 22.41 (t), 14.00 (q, C(8)). MS (EI): 246 (57), 245 (23), 231 (9), 215 (42), 203 (14), 189 (19), 175 (13), 161 (16), 159 (32), 147 (33), 131 (9), 121 (42), 115 (25), 108 (12), 103 (13), 91 (19), 43 (100).

### EXAMPLE 52

### (E)-3-(4-methoxybenzylidene)octan-2-one (Compound ID 36)

Prepared as described in Example 6 in 17% yield from 4-methoxybenzaldehyde and diethyl 2-oxooctan-3-ylphosphonate (obtained from hexyl iodide and triethyl phosphite via diethyl hexylphosphonate). Boiling point: 145° C. (0.06 mbar).

¹³C-NMR (100 MHz, CDCl₃): δ 200.16 (s, C(2)), 159.92 (s, C(3)), 141.19 (s), 139.26 (d, CH═C(3)), 131.12 (d, 2 C), 128.25 (s), 114.04 (d, 2 C), 55.30 (q, OMe), 32.13 (t), 28.68 (t), 26.28 (t), 26.05 (q, C(1)), 22.44 (t), 14.03 (q, C(8)). MS (EI): 246 (17), 231 (11), 215 (12), 203 (2), 189 (15), 175 (5), 161 (8), 159 (14), 147 (22), 132 (6), 121 (29), 108 (30), 43 (100).

### EXAMPLE 53

### (E)-3-(4-methoxybenzylidene)heptan-2-one (Compound ID 37)

Prepared as described in Example 48 in 24% yield from 4-methoxybenzaldehyde and 2-heptanone. Boiling point: 140° C. (0.08 mbar).

¹³C-NMR (100 MHz, CDCl₃): δ 200.15 (s, C(2)), 159.93 (s, C(3)), 141.15 (s), 139.28 (d, CH═C(3)), 131.13 (d, 2 C), 128.25 (s), 114.04 (d, 2 C), 55.29 (q, OMe), 31.15 (t), 28.68 (t), 26.05 (t), 26.04 (q, C(1)), 23.03 (t), 13.88 (q, C(7)). MS (EI): 231 (16), 217 (37), 201 (37), 189 (45), 175 (7), 161 (18), 159 (28), 147 (52), 132 (14), 121 (39), 115 (23), 108 (45), 103 (16), 91 (18), 77 (16), 43 (100).

### EXAMPLE 54

### (E)-3-(4-methoxybenzylidene)hexan-2-one (Compound ID 38)

Prepared as described in Example 48 in 35% yield from 4-methoxybenzaldehyde and 2-hexanone. Boiling point: 134° C. (0.08 mbar).

¹³C-NMR (100 MHz, CDCl₃): δ 200.16 (s, C(2)), 159.93 (s, C(3)), 141.02 (s), 139.43 (d, CH═C(3)), 131.13 (d, 2 C), 128.24 (s), 114.06 (d, 2 C), 55.30 (q, OMe), 28.29 (t), 26.03 (q, C(1)), 22.30 (t), 14.33 (q, C(6)). MS (EI): 218 (63), 203 (53), 189 (43), 187 (46), 175 (26), 161 (14), 160 (14), 159 (10), 147 (32), 132 (14), 121 (30), 115 (24), 108 (26), 103 (20), 91 (18), 77 (19), 43 (100).

### EXAMPLE 55

### (E)-3-(benzo[d][1,3]dioxol-5-ylmethylene)octan-2-one (Compound ID 39)

Prepared as described in Example 35 in 14% yield from Heliotropine and diethyl 2-oxooctan-3-ylphosphonate (obtained from hexyl iodide and triethyl phosphite via diethyl hexylphosphonate)._Boiling point: 145° C. (0.08 mbar).

¹³C-NMR (100 MHz, CDCl₃): δ 200.07 (s, C(2)), 147.95 (s, 2 C), 144.65 (s), 139.17 (d, CH═C(3)), 129.79 (s), 124.38 (d), 109.11 (d), 108.44 (d), 101.37 (t, OCH₂O), 32.07 (t), 28.67 (t), 26.27 (t), 26.07 (q, C(1)), 22.39 (t), 14.00 (q, C(8)). MS (EI): 260 (58), 245 (12), 230 (8), 217 (8), 203 (35), 190 (3), 189 (10), 187 (11), 173 (45), 161 (10), 160 (10), 159 (13), 145 (27), 135 (43), 131 (55), 122 (50), 115 (20), 103 (24), 77 (15), 43 (100).

### EXAMPLE 56

### (Z)-methyl 4-(2-acetylhept-1-enyl)benzoate (Compound ID 41) and (E)-methyl 4-(2-acetylhept-1-enyl)benzoate (Compound ID 40)

Prepared as described in Example 41 from methyl-4-formylbenzoate (5.59 g, 34 mmol) and diethyl 2-oxooctan-3-ylphosphonate (6 g, 22.7 mmol, obtained from hexyl iodide and triethyl phosphite via diethyl hexylphosphonate). Ball-to-ball distillation (0.08 mbar) of the crude product (8.4 g) followed by FC (280 g SiO₂, hexane/methyl t-butyl ether 5:1) of the fraction distilling at 177° C. (4.3 g) gave (Z)-methyl 4-(2-acetylhept-1-enyl)benzoate (0.56 g, 9%) and (E)-methyl 4-(2-acetylhept-1-enyl)benzoate (3.15 g, 34%).

(Z)-methyl 4-(2-acetylhept-1-enyl)benzoate:

Boiling point: 150° C. (0.08 mbar).

¹H-NMR (400 MHz, CDCl₃): δ 7.98 (dt, J=1.8, 8.3, 2H), 7.26 (dm, J=8.5, 2H), 6.61 (br. s, HC═C(3)), 3.91 (s, OMe), 2.41-2.35 (m, 2H—C(4)), 2.04 (s, C(1)H₃), 1.55-1.45 (m, 2H), 1.38-1.31 (m, 4H), 0.91 (t, J=7.1, C(8)H₃). ¹³C-NMR (100 MHz, CDCl₃): δ 207.47 (s), 166.63 (s), 147.04 (s), 140.95 (s), 129.72 (d, 2 C), 129.32 (s), 128.38 (d), 128.24 (d, 2 C), 52.10 (q, OMe), 35.52 (t), 31.38 (t), 30.77 (q, MeCO), 27.70 (t), 22.38 (t), 13.95 (q, C(7)). MS (EI): 274 (7), 259 (29), 243 (7), 231 (2), 215 (100), 203 (5), 189 (5), 175 (4), 159 (13), 149 (9), 143 (16), 129 (14), 115 (25), 91 (9), 59 (11), 43 (36).

(E)-methyl 4-(2-acetylhept-1-enyl)benzoate:

Boiling point: 150° C. (0.08 mbar).

¹H-NMR (400 MHz, CDCl₃): δ 8.08 (dt, J=1.8, 8.3, 2H), 7.46 (br. s, HC═C(3)), 7.43 (dm, J=8.5, 2H), 3.94 (s, OMe), 2.50-2.44 (m, 2H-C(4)), 2.45 (s, C(1)H₃), 1.49-1.38 (m, 2H), 1.35-1.26 (m, 4H), 0.87 (t, J=7.1, C(8)H₃). ¹³C-NMR (100 MHz, CDCl₃): δ 199.95 (s), 166.56 (s), 144.65 (s), 140.44 (s), 137.79 (d), 129.79 (s), 129.71 (d, 2 C), 128.99 (d, 2 C), 52.18 (q, OMe), 31.96 (t), 28.83 (t), 26.46 (t), 26.19 (q, MeCO), 22.31 (t), 13.94 (q, C(7)). MS (EI): 274 (5), 259 (24), 243 (6), 231 (2), 215 (100), 203 (5), 189 (4), 174 (4), 159 (13), 149 (9), 143 (15), 129 (14), 115 (25), 91 (9), 59 (12), 43 (44).

### EXAMPLE 57

### (E)-3-(thiophen-2-ylmethylene)octan-2-one (Compound ID 42)

Prepared as described in Example 35 in 29% yield from 3-thiophencarboxaldehyde and diethyl 2-oxooctan-3-ylphosphonate (obtained from hexyl iodide and triethyl phosphite via diethyl hexylphosphonate). Boiling point: 135° C. (0.08 mbar).

¹³C-NMR (100 MHz, CDCl₃): δ 199.88 (s, C(2)), 141.24 (s), 137.13 (s), 133.05 (d), 128.58 (d), 127.18 (d), 126.08 (d), 32.18 (t), 28.41 (t), 26.60 (t), 25.93 (q, C(1)), 22.51 (t), 14.03 (q, C(8)). MS (EI): 222 (45), 207 (10), 189 (2), 179 (30), 166 (3), 165 (14), 151 (11), 137 (19), 135 (17), 123 (50), 109 (19), 97 (39), 84 (4), 43 (100).

### EXAMPLE 58

### (E)-3-(pyridin-2-ylmethylene)octan-2-one (Compound ID 43)

Prepared as described in Example 48 in 44% yield from 2-pyridinecarboxaldehyde and 2-octanone. Boiling point: 120° C. (0.06 mbar).

¹³C-NMR (100 MHz, CDCl₃): δ 200.60 (s, C(2)), 155.03 (s), 149.71 (d), 145.73 (s), 137.29 (d), 136.22 (d), 125.08 (d), 122.62 (d), 32.02 (t), 28.71 (t), 26.30 (q, C(1)), 26.13 (t), 22.32 (t), 13.96 (q, C(8)). MS (EI): 217 (12), 202 (10), 188 (32), 175 (32), 174 (100), 160 (12), 146 (12), 145 (9), 144 (14), 132 (38), 131 (22), 130 (37), 118 (51), 117 (50), 106 (7), 93 (20), 78 (12), 43 (20).

### EXAMPLE 59

### (E)-3-(pyridin-3-ylmethylene)octan-2-one (Compound ID 44)

Prepared as described in Example 41 in 33% yield from 3-pyridinecarboxaldehyde and diethyl 2-oxooctan-3-ylphosphonate (obtained from hexyl iodide and triethyl phosphite via diethyl hexylphosphonate)._Boiling point: 115° C. (0.08 mbar).

¹³C-NMR (100 MHz, CDCl₃): δ 199.63 (s, C(2)), 150.09 (d), 149.23 (d), 145.07 (s), 135.99 (d), 135.04 (d), 131.69 (s), 123.32 (d), 31.94 (t), 28.86 (t), 26.48 (t), 26.16 (q, C(1)), 22.33 (t), 13.92 (q, C(8)). MS (EI): 217 (16), 216 (15), 202 (25), 188 (22), 175 (23), 174 (59), 160 (23), 146 (32), 132 (55), 130 (28), 118 (100), 117 (42), 106 (13), 92 (20), 91 (17), 89 (14), 43 (61).

### EXAMPLE 60

### (E)-3-(pyridin-4-ylmethylene)octan-2-one (Compound ID 45)

Prepared as described in Example 41 in 32% yield from 4-pyridinecarboxaldehyde and 2-octanone. Boiling point: 135° C. (0.08 mbar).

¹³C-NMR (100 MHz, CDCl₃): δ 199.64 (s, C(2)), 150.05 (d, 2 C), 146.15 (s), 143.56 (s), 135.62 (d), 123.22 (d, 2 C), 31.89 (t), 28.86 (t), 26.50 (t), 26.20 (q, C(1)), 22.28 (t), 13.90 (q, C(8)). MS (EI): 217 (49), 216 (10), 202 (22), 188 (22), 184 (2), 175 (18), 174 (88), 160 (23), 146 (43), 132 (64), 130 (46), 118 (100), 117 (55), 106 (16), 93 (17), 91 (25), 89 (19), 43 (89).

### EXAMPLE 61

### (E)-methyl 2-(cyclopropylmethylene)heptanoate (Compound ID 46)

At −75° C., a solution of diisopropylamine (7.7 ml, 54.1 mmol) in tetrahydrofuran (60 ml) was treated with a 1.6M solution of n-butyllithium in hexane (34 ml, 54.1 mmol). The resulting solution was stirred for 30 min. at −75° C. and treated with a solution of methyl heptanoate (6.0 g, 41.6 mmol) in tetrahydrofuran (20 ml). The resulting solution was stirred for 30 min. at −75° C. and treated with a solution of cyclopropanecarboxaldehyde (12.7 ml, 166.4 mmol) in tetrahydrofuran (20 ml). After stirring for 2 h at −75° C., the reaction mixture was poured into ice-cold 2M aqueous HCl (50 ml) and extracted twice with methyl t-butyl ether (100 ml). The combined organic phases were washed with water (50 ml), aqueous NaCl solution (50 ml), dried (MgSO₄), and the solvent evaporated to give an oil (9.66 g). A part of this residue (4.83 g) was treated with acetic anhydride (4.5 ml, 47.3 mmol) and sodium acetate (2.04 g, 24.8 mmol). The resulting mixture was stirred for 32 h at 80° C. and for 65 h at 20° C., poured into an ice-cold 2M NaOH solution (50 ml) and extracted twice with methyl t-butyl ether (50 ml). The combined organic phases were washed with a saturated aqueous solution of NaHCO₃ (25 ml), water (25 ml), aqueous NaCl solution (25 ml), dried (MgSO₄), and the solvent evaporated to give an oil (5.28 g). A solution of a part of this residue (2.6 g) in toluene (20 ml) was treated at 20° C. with a solution of DBU (3.1 ml, 20.3 mmol) in toluene (5 ml). The resulting solution was stirred for 1 h at 20° C., 1 h at 50° C. and 28 h at reflux, poured into ice-cold 2M aqueous HCl (50 ml), and extracted twice with methyl t-butyl ether (50 ml). The combined organic phases were washed with water (50 ml), aqueous NaCl solution (50 ml), dried (MgSO₄), and the solvent evaporated. FC (100 g SiO₂, hexane/methyl t-butyl ether 60:1) of the crude product (2.1 g) gave (E)-methyl 2-(cyclopropylmethylene)heptanoate (0.6 g, 29%). Boiling point: 95° C. (0.07 mbar).

¹H-NMR (400 MHz, CDCl₃): δ 6.10 (d, J=10.6, H—C═C(2)), 3.71 (s, OMe), 2.39 (br. t, J=7.7, 2H—C(3)), 1.67-1.57 (m, H—CCH═), 1.50-1.25 (m, C(4)H₂, C(5)H₂, C(6)H₂), 0.94 (ddd, J=4.3, 6.6, 7.8, 2H), 0.89 (t, J=7.1, C(7)H₃), 0.59 (dt, J=4.5, 6.8, 2H). ¹³C-NMR (100 MHz, CDCl₃): δ 168.37 (s, C(1)), 147.65 (d, CH═C(2)), 130.03 (s, C(2)), 51.44 (q, OMe), 31.69 (t), 29.14 (t), 26.82 (t), 22.52 (t), 14.02 (q, C(7)), 11.42 (d), 8.35 (t, 2 C) MS (EI): 196 (12), 181 (21), 168 (90), 165 (15), 153 (3), 139 (20), 125 (30), 111 (65), 107 (38), 95 (30), 93 (27), 91 (15), 81 (44), 79 (100), 77 (37), 67 (50), 59 (33), 55 (37), 53 (24), 41 (41).

### EXAMPLE 62

### (E)-methyl 2-benzylideneheptanoate (Compound ID 47)

Prepared as described in Example 61 in 48% yield from methyl heptanoate and benzaldehyde. Boiling point: 140° C. (0.08 mbar).

¹³C-NMR (100 MHz, CDCl₃): δ 168.99 (s, C(1)), 138.70 (d, CH═C(2)), 135.85 (s), 133.72 (s), 129.18 (d, 2 C), 128.42 (d, 2 C), 128.25 (d), 51.90 (q, OMe), 31.88 (t), 28.92 (t), 27.50 (t), 22.35 (t), 13.98 (q, C(7)). MS (EI): 232 (30), 217 (1), 201 (9), 200 (6), 175 (5), 172 (13), 162 (8), 161 (7), 158 (7), 143 (15), 131 (28), 130 (34), 129 (32), 128 (18), 121 (14), 117 (38), 116 (48), 115 (100), 104 (7), 91 (47), 77 (10), 59 (15).

### EXAMPLE 63

### methyl 2-(cyclopropylmethyl)heptanoate (Compound ID 48)

A solution of (E)-methyl 2-(cyclopropylmethylene)heptanoate (0.95 g, 4.8 mmol, prepared as described in Example 61) in ethanol (30 ml) was treated with Lindlar catalyst (0.6 g), quinoline (1.2 ml, 8.6 mmol), and triethylamine (0.8 ml, 6.8 mmol), and the resulting mixture hydrogenated for 72 h (5 bar). After filtration, the reaction mixture was poured into ice (50 g) and 2M aqueous HCl (20 ml) and extracted twice with methyl t-butyl ether (50 ml). The combined organic phases were washed with water (50 ml), is aqueous NaCl solution (25 ml), dried (MgSO₄), and the solvent evaporated. FC (90 g SiO₂, pentane/diethyl ether 60:1) of the crude product (0.8 g) gave methyl 2-(cyclopropylmethyl)heptanoate (0.39 g, 41%). Boiling point: 90° C. (0.08 mbar).

¹³C-NMR (100 MHz, CDCl₃): δ 176.95 (s, C(1)), 51.26 (q, OMe), 46.16 (d), 37.53 (t), 32.25 (t), 31.71 (t), 27.09 (t), 22.45 (t), 13.96 (q, C(7)), 9.06 (d), 4.40 (t), 4.26 (t). MS (EI): 198 (1), 183 (1), 167 (1), 155 (6), 149 (4), 141 (100), 128 (14), 113 (11), 109 (30), 101 (19), 87 (75), 81 (20), 74 (7), 69 (16), 68 (11), 67 (15), 59 (15), 55 (66), 41 (31).

### EXAMPLE 64

### methyl 2-benzylheptanoate (Compound ID 49)

A mixture of (E)-methyl 2-benzylideneheptanoate (1.1 g, 4.7 mmol, prepared as described in Example 62) and 10% Pd/C (0.29 g) in ethanol (40 ml) was hydrogenated for 1.5 h (5 bar). After filtration and solvent evaporation, FC (90 g SiO₂, hexane/methyl t-butyl ether 40:1) of the crude product (1.14 g) gave methyl 2-benzylheptanoate (1.09 g, 98%). Boiling point: 120° C. (0.08 mbar).

¹³C-NMR (100 MHz, CDCl₃): δ 176.16 (s, C(1)), 139.48 (s), 128.81 (d, 2 C), 128.32 (d, 2 C), 126.24 (d), 51.31 (q, OMe), 47.68 (d, C(2)), 38.53 (t), 32.08 (t), 31.64 (t), 26.99 (t), 22.45 (t), 13.96 (q, C(7)). MS (EI): 234 (8), 174 (28), 164 (16), 163 (18), 131 (28), 117 (16), 104 (27), 91 (100), 78 (6), 65 (8).

### EXAMPLE 65

### 3-(cyclopropylmethyl)octan-2-one (Compound ID 50)

A solution of (E)-3-(cyclopropylmethylene)octan-2-one (1.6 g, 8.9 mmol, prepared as described in Example 6) in ethanol (30 ml) was treated with Lindlar catalyst (0.49 g), quinoline (1.0 ml, 7.2 mmol), and triethylamine (1.5 ml, 12.7 mmol), and the resulting mixture hydrogenated for 6 h (1 bar). After filtration, the reaction mixture was poured into 2M aqueous HCl (30 ml) and extracted twice with cyclohexane (60 ml). The combined organic phases were washed with water (60 ml), aqueous NaCl solution (60 ml), dried (MgSO₄), and the solvent evaporated. FC (200 g SiO₂, hexane/methyl t-butyl ether 40:1) of the crude product (1.7 g) gave 3-(cyclopropylmethyl)octan-2-one (1.1 g, 68%). Boiling point: 80° C. (0.08 mbar).

¹³C-NMR (100 MHz, CDCl₃): δ 213.30 (s, C(2)), 53.54 (d, C(3)), 36.95 (t), 31.89 (t), 31.71 (t), 29.44 (q, C(1)), 27.05 (t), 22.43 (t), 13.95 (q, C(8)), 9.16 (d), 4.84 (t), 4.54 (t). MS (EI): 182 (1),167 (1), 153 (2), 139 (3),125 (78), 112 (11), 111 (7), 107 (4), 97 (16), 83 (18), 71 (26), 69 (13), 67 (10), 55 (51), 43 (100), 41 (25).

### EXAMPLE 66

### (E)-3-(1-phenylethylidene)octan-2-one (Compound ID 51)

At 23° C., a solution of sodium bromide (13.0 g, 124.8 mmol) in DMF (230 ml) was treated within 5 min. with a solution of trimethylchlorosilane (16.1 ml, 124.8 mmol) in DMF (20 ml), then within 10 min. with a solution of triethylamine (17.7 ml, 124.8 ml) in DMF (20 ml), and then with a solution of 2-octanone (10.0 g, 78.0 mmol) in DMF (30 ml). The resulting mixture was stirred at 23° C. for 48 h, poured into ice-cold water (100 ml), and extracted twice with hexane (200 ml). The combined organic phases were washed with a saturated aqueous NaHCO₃ solution (50 ml), twice with water (100 ml), dried (MgSO₄), and the solvent evaporated. Ball-to-ball distillation (77-110° C., 0.08 mbar) of the crude product (13 g) gave a fraction of silyl enol ethers (10.4 g) that was dissolved in part (1.0 g, 4.9 mmol) in 1,2-dichloroethane (3.5 ml), and treated with tin tetrachloride (0.6 ml, 5.1 mmol). The resulting solution was stirred for 10 min. and added dropwise to a mixture obtained by treating within 5 min. a solution of tin tetrachloride (0.4 ml, 3.4 mmol) in acetonitrile (8.0 ml) with a solution of phenyl acetylene (0.38 ml, 3.3 mmol) and tributylamine (1.1 ml, 3.3 mmol) in acetonitrile (5 ml) and stirring the yellow mixture for 30 min. The orange reaction mixture was refluxed for 1.5 h, and poured into an ice-cold saturated aqueous NaHCO₃ solution (100 ml). The resulting mixture was filtered, washed with cyclohexane, and the filtrate was extracted twice with cyclohexane (50 ml). The combined organic phases were washed twice with water (50 ml), twice with aqueous NaCl solution (50 ml), dried (MgSO₄), and the solvent evaporated. FC (100 g SiO₂, hexane/methyl t-butyl ether 40:1) of the crude product (1.4 g) gave 3-(cyclopropylmethyl)octan-2-one (0.21 g, 7%). Boiling point: 125° C. (0.08 mbar).

¹³C-NMR (100 MHz, CDCl₃): δ 207.28 (s, C(2)), 142.79 (s), 139.66 (s), 137.78 (s), 128.27 (d, 2 C), 127.37 (d, 2 C), 126.89 (d), 31.49 (t), 30.71 (t), 30.31 (q), 28.58 (t), 22.61 (q, C(1)), 22.19 (t), 13.85 (q, C(8)). MS (EI): 230 (36), 229 (40), 215 (39), 197 (1), 173 (24), 159 (20), 145 (15), 131 (41), 129 (21), 128 (19), 117 (13), 115 (24), 105 (26), 91 (33), 43 (100).

### EXAMPLE 67

### (E)-2-(cyclopropylmethylene)-1-phenylheptan-1-one (Compound ID 52)

At −75° C., a solution of diisopropylamine (5.8 ml, 41.0 mmol) in tetrahydrofuran (60 ml) was treated with a 1.6M solution of n-butyllithium in hexane (26 ml, 41.0 mmol). The resulting solution was stirred 30 min. at −70° C. and treated with a solution of heptanophenone (6.0 g, 31.5 mmol) in tetrahydrofuran (20 ml). The resulting solution was stirred for 30 min. at −70° C. and treated with a solution of cyclopropanecarboxaldehyde (9.6 ml, 126.1 mmol) in tetrahydrofuran (20 ml). After stirring for 2 h at −75° C., the reaction mixture was poured into ice-cold 2M aqueous HCl (50 ml), and extracted twice with methyl t-butyl ether (100 ml). The combined organic phases were washed with water (50 ml), aqueous NaCl solution (50 ml), dried (MgSO₄), and the solvent evaporated to give an oil (8.73 g) that was treated with acetic anhydride (6.7 ml, 70.4 mmol) and sodium acetate (3.03 g, 36.9 mmol). The resulting mixture was stirred for 54 h at 80° C. and for 22 h at 120° C., poured into an ice-cold 2M NaOH solution (50 ml), and extracted twice with methyl t-butyl ether (50 ml). The combined organic phases were washed with a saturated aqueous solution of NaHCO₃ (25 ml), water (25 ml), aqueous NaCl solution (25 ml), dried (MgSO₄), and the solvent evaporated. FC (300 g SiO₂, hexane/methyl t-butyl ether 60:1) of the crude product (7.0 g) gave (E)-2-(cyclopropylmethylene)-1-phenylheptan-1-one (5.9 g, 77%). Boiling point: 145° C. (0.07 mbar).

¹³C-NMR (100 MHz, CDCl₃): δ 198.13 (s, C(1)), 151.91 (d, CH═C(2)), 139.44 (s), 139.37 (s), 131.01 (d), 129.14 (d, 2 C), 127.94 (d, 2 C), 31.89 (t), 28.88 (t), 26.71 (t), 22.56 (t), 14.06 (q, C(7)), 11.83 (d), 8.77 (t, 2 C). MS (EI): 242 (5), 241 (2), 227 (6), 214 (12), 199 (2), 185 (9), 172 (19), 171 (11), 157 (25), 129 (12), 122 (4), 115 (5), 105 (100), 91 (9), 77 (58).

### EXAMPLE 68

### (E)-methyl 2-(2,2-dimethylpropylidene)heptanoate (Compound ID 53)

Prepared as described in Example 61 in 38% yield from methyl heptanoate and pivalaldehyde. Boiling point: 75° C. (0.07 mbar).

¹³C-NMR (100 MHz, CDCl₃): δ 169.57 (s, C(1)), 150.74 (d, CH═C(2)), 131.75 (s, C(2)), 51.64 (q, OMe), 33.30 (s), 32.20 (t), 30.57 (q, Me₃C), 29.63 (t), 27.40 (t), 22.42 (t), 13.99 (q, C(7)). MS (EI): 212 (6), 197 (18), 181 (10), 165 (5), 155 (100), 141 (9), 123 (55), 109 (15), 95 (65), 81 (29), 73 (35), 69 (16), 67 (20), 59 (10), 57 (13), 55 (33), 53 (12), 41 (29).

### EXAMPLE 69

### (E)-2-(2,2-dimethylpropylidene)heptanoic acid (Compound ID 54)

A mixture of (E)-methyl 2-(2,2-dimethylpropylidene)heptanoate (4 g, 18.8 mmol, prepared as described in Example 68) and KOH (12.4 g, 188 mmol) in ethanol (80 ml) was refluxed for 2 h, poured into ice-cold 2M aqueous HCl (50 ml) and extracted twice with methyl t-butyl ether (80 ml). The combined organic phases were washed with water (80 ml), aqueous NaCl solution (80 ml), dried (MgSO₄), and the solvent evaporated. FC (300 g SiO₂, hexane/methyl t-butyl ether 7:1) of the crude product (3.76 g) gave a fraction (2 g) that was stirred in concentrated aqueous NaOH for 20 min. Extraction with hexane followed by acidification of the aqueous phase with concentrated aqueous HCl and extraction with methyl t-butyl ether, and washing of the resulting organic phases with water gave after solvent evaporation (E)-2-(2,2-dimethylpropylidene)heptanoic acid (1.6 g, 43%).

¹³C-NMR (100 MHz, CDCl₃): δ 174.65 (s, C(1)), 153.29 (d, CH═C(2)), 131.03 (s, C(2)), 33.52 (s), 32.24 (t), 30.43 (q, Me₃C), 29.64 (t), 27.07 (t), 22.44 (t), 14.02 (q, C(7)). MS (EI): 198 (5), 183 (15), 165 (2), 155 (2), 141 (100), 139 (9), 123 (39), 109 (11), 95 (50), 81 (30), 69 (23), 67 (19), 59 (36), 55 (42), 41 (39).

### EXAMPLE 70

### (E)-3-(2,2-dimethylpropylidene)octan-2-one (Compound ID 55)

At −30° C., a solution of (E)-2-(2,2-dimethylpropylidene)heptanoic acid (1.4 g, 7.06 mmol, prepared as described in Example 69) in diethyl ether (30 ml) was treated dropwise within 10 min. with a 1.6M solution of methyllithium in hexane (9.2 ml, 14.8 mmol). The resulting mixture was diluted with diethyl ether (15 ml), slowly warmed during 2 h to 20° C., poured into ice-cold 2M aqueous HCl (40 ml), and extracted twice with diethyl ether (40 ml). The combined organic phases were washed with water (40 ml), aqueous NaCl solution (40 ml), dried (MgSO₄), and the solvent evaporated. FC (120 g SiO₂, hexane/methyl t-butyl ether 30:1) of the crude product (1.2 g) gave (E)-3-(2,2-dimethylpropylidene)octan-2-one (0.83 g, 60%). Boiling point: 66° C. (0.08 mbar).

¹³C-NMR (100 MHz, CDCl₃): δ 201.13 (s, C(2)), 151.84 (d, CH═C(2)), 141.84 (s, C(2)), 33.51 (s), 32.38 (t), 30.63 (q, Me₃C), 29.61 (t), 26.14 (t), 26.11 (q, C(1)), 22.45 (t), 14.01 (q, C(8)). MS (EI): 196 (8), 181 (25), 163 (1), 153 (4), 139 (100), 125 (8), 121 (32), 111 (12), 97 (15), 83 (23), 69 (19), 57 (18), 55 (31), 43 (92).

### EXAMPLE 71

### (E)-3-(2-methylpropylidene)octan-2-one (Compound ID 56)

Prepared as described in Example 35 in 14% yield from isobutyraldehyde and diethyl 2-oxooctan-3-ylphosphonate (obtained from hexyl iodide and triethyl phosphite via diethyl hexylphosphonate). Boiling point: 47° C. (0.078 mbar).

¹³C-NMR (100 MHz, CDCl₃): δ 200.03 (s, C(2)), 150.07 (d, CH═C(3)), 140.04 (s, C(3)), 31.98 (d), 29.45 (t), 28.18 (d), 25.65 (q, C(1)), 25.54 (t), 22.48 (t), 22.37 (q, 2 C), 13.97 (q, C(8)). MS (EI): 182 (19), 167 (6), 149 (1), 139 (25), 125 (100), 112 (3), 111 (7), 107 (13), 97 (12), 83 (16), 69 (37), 55 (30), 43 (97).

### EXAMPLE 72

### (E)-4-(cyclopropylmethylene)nonan-3-one (Compound ID 57)

Diethyl 3-oxononan-4-ylphosphonate was prepared in 93% yield from ethyl propionate and diethyl pentylphosphonate as described in Example 6. Boiling point: 106° C. (0.06 mbar).

¹³C-NMR (100 MHz, CDCl₃): δ 206.56 (s, J=4.6, CO), 62.50 (t, J=6.6, CH₂O), 62.37 (t, J=6.6, CH₂O), 52.68 (d, J=125.2, C(4)), 37.44 (t, C(2)), 31.43 (t), 28.20 (t, J=14.9, C(6)), 26.48 (t, J=5.4, C(5)), 22.25 (t), 16.34 (q, J=2.1, MeCH₂O), 16.28 (q, J=2.1, MeCH₂O), 13.87 (q, C(9)), 7.58 (q, C(1)). MS (EI): 278 (1), 263 (1), 249 (18), 233 (3), 222 (29), 221 (16), 208 (34), 193 (11), 179 (56), 165 (100), 152 (30), 138 (18), 137 (18), 123 (16), 109 (34), 91 (12), 83 (14), 81 (16), 65 (10), 57 (22), 55 (24), 41 (13), 29 (19).

(E)-4-(cyclopropylmethylene)nonan-3-one was prepared as described in Example 35 in 38% yield from benzaldehyde and diethyl 3-oxooctan-4-ylphosphonate.

Boiling point: 90° C. (0.07 mbar).

¹³C-NMR (100 MHz, CDCl₃): δ 201.32 (s, C(3)), 147.30 (d, CH═C(4)), 139.86 (s, C(4)), 31.92 (t), 30.12 (t), 29.15 (t), 25.87 (t), 22.53 (t), 14.02 (q, C(9)), 11.61 (d), 8.89 (q, C(1)), 8.62 (t, 2 C). MS (EI): 194 (2), 179 (11), 166 (40), 165 (82), 151 (6), 137 (19), 123 (22), 110 (54), 109 (57), 95 (48), 81 (85), 67 (63), 57 (100).

### EXAMPLE 73

### (E)-4-benzylidenenonan-3-one (Compound ID 58)

Prepared as described in Example 35 in 12% yield from benzaldehyde and diethyl 3-oxononan-4-ylphosphonate (obtained as described in Example 77 from ethyl propionate and diethyl pentylphosphonate). Boiling point: 130° C. (0.07 mbar).

¹³C-NMR (100 MHz, CDCl₃): δ 203.09 (s, C(3)), 142.68 (s, C(4)), 137.75 (d, CH═C(4)), 135.98 (s), 129.16 (d, 2 C), 128.48 (d, 2 C), 128.31 (d), 32.05 (t), 31.00 (t), 28.90 (t), 26.62 (t), 22.36 (t), 13.99 (q, C(9)), 8.84 (q, C(1)). MS (EI): 230 (30), 229 (9), 215 (1), 201 (100), 173 (9), 159 (6), 145 (6), 131 (15), 129 (21), 117 (80), 115 (45), 105 (12), 91 (75), 57 (49).

### EXAMPLE 74

### Inhibition of Human CYP2B6

Test compounds that inhibit the activity of CYP2B6 are identified by using the same principle as described in Example 1, first paragraph.

A test compound (details see Table 4) was incubated with CYP2B6 in the presence of a cytochrome P450 reductase. CYP2B6 and P450 reductase are produced using recombinant baculoviruses and co-expressing the two proteins in Sf9 insect cells as described in Example 1. Alternatively, microsomes containing CYP2B6 and the reductase are commercially available (BD Biosciences Gentest, USA). Microsomes were used which contained 1.5 pmoles CYP2B6. Potassium phosphate buffer final concentration was 100 mM, (1M stock, pH 7.4). The test compound was prepared as a 50 mM stock solution in acetonitrile. The concentration of the standard substrate 7-ethoxy-4-trifluoromethyl-coumarin was 6 μM. Several samples of the test compound were prepared at various concentrations to give different final concentrations in the reaction: 0, 0.005, 0.01, 0.02, 0.05, 0.1 and 0.2 mM. (As obvious to the person skilled in the art, in cases where very good inhibitors were tested, lower concentrations were also used in order to have concentrations above and below the IC50 concentration present in the test wells.) The mixture was incubated for 10 min at 37° C. prior to the initiation of the enzymatic reaction by the addition of 0.005 ml of a solution of 50 mM NADPH in water. The final total volume was 0.2 ml, which is suitable for microtiter plate measurements. The samples were incubated for 40 min at 37° C. After 40 min, the enzymatic reaction was stopped by the addition of 75 μl of 0.5M Tris-base/acetonitrile (18:72). 0.005 ml of a solution of 50 mM NADPH in water was added to the control reaction which corresponds to the reaction with test compound and enzyme but without NADPH, and as a consequence, no 4-trifluoromethyl-umbelliferone was formed. Denatured proteins and other insoluble parts were separated by centrifugation (5 min, 1800 rpm, at 10° C.).

The samples were analysed spectrofluorometrically which allows to detect the formation of 4-trifluoromethyl-umbelliferone as the enzymatic product at an excitation wavelength of 410 nm and an emission wavelength of 510 nm. A decrease of the fluorescent signal at 510 nm with respect to the control shows that the test compound is influencing enzymatic activity and confirms the nature of an inhibitor, which can also be an alternative substrate. Graphical analysis of the data allows to calculate the concentration, where the test compound inhibits the enzyme to the level of 50% maximal activity (IC50 value). The results are shown in Table 4 below.

TABLE 4
CYP2B6 inhibitor activity

| Compound | |
|---|---|
| IC₅₀ (μM) | Chemical Structure |
| ID 1 3.9 μM | 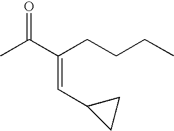 |
| ID 2 3.5 μM | 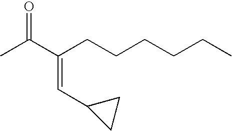 |
| ID 3 4.0 μM | |

1. A composition comprising
a) a compound of formula (I) 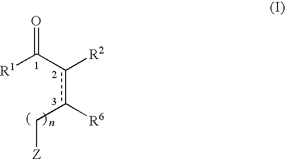
wherein n is 0 or 1;
the dotted line represents together with the carbon-carbon bond a double bond, either in E or Z configuration, or a single bond;
R¹ is C₁-C₃ alkyl, C₃-C₇ alkenyl, cycloalkylvinyl comprising from 5 to 7 carbon atoms, arylvinyl comprising from 5 to 7 carbon atoms, phenyl, hydroxyl, C₁-C₃ alkoxy, C₂-C₃ alkenyloxy, or ethinyl;
R² is linear or branched C₃-C₇ alkyl;
I) Z is —CR³R⁴R⁵ wherein R³, R⁴, R⁵ are hydrogen; R³ and R⁴ are methyl and R⁵ is hydrogen or methyl; or R³ and R⁴ representing independently H, or C₁-C₆ alkoxy and R⁵ is C₁-C₆ alkoxy;
II) Z is a 3-6 membered monocyclic or 6-10 membered bicyclic hydrocarbon ring wherein up to two, C atom(s) are replaced by a hetero atom selected from S, O, and N;
III) Z is a 3-6 membered monoyclic or 6-10 membered bicyclic hydrocarbon ring wherein up to two, C atom(s) are replaced by a hetero atom selected from S, O, and N, and the ring is substituted with up to 5 groups selected from hydroxyl, CN, halogen, mono-, di-, and trihalogenomethyl, C₁-C₃ alkoxy, C₁-C₃ alkyl and —COOR, and —OCOR, wherein R is hydrogen, methyl, ethyl, propyl or isopropyl, with the proviso that the ring is substituted with up to one C₁-C₃ alkyl group;
IV) Z is a bivalent residue —CH₂—CH₂— forming together with the C-2 a cyclobutan and cyclopentan ring respectively; or
V) Z is —C(O)R⁷ wherein R⁷ is C₁-C₃ alkyl, or C₁-C₃ alkoxy;
R⁶ is H, C₁-C₃ alkyl, or —CH₂— forming with C-2 a cyclopropan ring; and
the compound of formula (I) contains at least 9 C-atoms;
b) and at least one odorant compound.

2. A composition according to claim 1 wherein the compound of formula (I) is selected from the group consisting of: (E)-3-(cyclopropylmethylene)octan-2-one; (E)-3-(cyclopropylmethylene)heptan-2-one; (E)-3-(cyclopropylmethylene)nonan-2-one; (1E,4E)-1-cyclopropyl-4-(cyclopropyl-methylene)dec-1-en-3-one; (E)-3-benzylideneheptan-2-one; (E)-3-benzylideneoctan-2-one; (1E,4E)-4-benzylidene-1-phenylnon-1-en-3-one; (E)-3-benzylidenenonan-2-one; 3-phenylmethylheptan-2-one; 3-phenylmethyloctan-2-one; (E)-4-(2-acetylhept-1-enyl)-benzonitrile; (E)-3-(naphthalen-2-ylmethylene)octan-2-one; (E)-3-(thiophen-2-ylmethylene)octan-2-one; (E)-3-(furan-2-ylmethylene)octan-2-one; 3-((tetrahydrofuran-2-yl)methyl)octan-2-one; (E)-3-((tetrahydrofuran-3-yl)methylene)heptan-2-one; (E)-3-((tetrahydrofuran-3-yl)methylene)octan-2-one; 3-((tetrahydrofuran-3-yl)methyl)octan-2-one; (E)-3-(2,2-dimethoxyethylidene)heptan-2-one; (E)-3-(2,2-dimethoxyethylidene)-octan-2-one; 3-(2,2-dimethoxyethyl)octan-2-one; 3-(2-methoxyethyl)octan-2-one; (E)-3-(2-(2-methyl-1,3-dioxolan-2-yl)ethylidene)octan-2-one; 3-(2-(2-methyl-1,3-dioxolan-2-yl)ethyl)octan-2-one; 3-pentylheptane-2,6-dione; (E)-3-ethylideneoctan-2-one; 142-methyl-1-pentylcyclopropyl)ethanone; 3-(propan-2-ylidene)octan-2-one; methyl 1-pentylcyclopentanecarboxylate; 1-(1-pentylcyclopentyl)ethanone; (E)-3-(cyclohexylmethylene)octan-2-one; (E)-3-(cyclohex-3-enylmethylene)octan-2-one; (E)-3-(cyclopentylmethylene)octan-2-one; (E)-3-(cyclobutylmethylene)octan-2-one; (E)-3-(2-fluorobenzylidene)octan-2-one; (E)-3-(3-fluorobenzylidene)octan-2-one; (E)-3-(4-fluorobenzylidene)octan-2-one; (E)-3-(2,6-difluorobenzylidene)octan-2-one; (E)-3-(2,4-difluorobenzylidene)octan-2-one; (Z)-3-(3,5-difluorobenzylidene)octan-2-one; (E)-3-(3,5-difluorobenzylidene)octan-2-one; (E)-3-(perfluorobenzylidene)octan-2-one; (E)-3-(2-methylbenzylidene)octan-2-one; (E)-3-(3-methylbenzylidene)octan-2-one; (E)-3-(4-methylbenzylidene)octan-2-one; (E)-3-(2-(trifluoromethyl)benzylidene)octan-2-one; (E)-3-benzylidenehexan-2-one; (E)-3-(2-methoxybenzylidene)octan-2-one; (E)-3-(3-methoxybenzylidene)octan-2-one; (E)-3-(4-methoxybenzylidene)octan-2-one; (E)-3-(4-methoxybenzylidene)heptan-2-one; (E)-3-(4-methoxybenzylidene)hexan-2-one; (E)-3-(benzo[d][1,3]dioxol-5-ylmethylene)octan-2-one; (Z)-methyl 4-(2-acetylhept-1-enyl)benzoate; (E)-methyl 4-(2-acetylhept-1-enyl)benzoate; (E)-3-(thiophen-2-ylmethylene)octan-2-one; (E)-3-(pyridin-2-ylmethylene)octan-2-one; (E)-3-(pyridin-3-ylmethylene)octan-2-one; (E)-3-(pyridin-4-ylmethylene)octan-2-one; (E)-methyl 2-(cyclopropylmethylene)heptanoate; (E)-methyl 2-benzylideneheptanoate; methyl 2-(cyclopropylmethyl)heptanoate; methyl 2-benzylheptanoate; 3-(cyclopropylmethyl)octan-2-one; (E)-3-(1-phenylethylidene)octan-2-one; (E)-2-(cyclopropylmethylene)-1-phenylheptan-1-one; (E)-methyl 2-(2,2-dimethylpropylidene)heptanoate; (E)-2-(2,2-dimethylpropylidene)heptanoic acid; (E)-3-(2,2-dimethylpropylidene)octan-2-one; (E)-3-(2-methylpropylidene)octan-2-one; (E)-4-(cyclopropylmethylene)nonan-3-one; (E)-4-benzylidenenonan-3-one; or a mixture thereof.

3. A tobacco product comprising a compound of formula (I) according to claim 1.

4. A method comprising the step of disseminating a compound of formula (I) according to claim 1 into a room in the presence of tobacco smoke.

5. A method according to claim 4 wherein the compound of formula (I) is diffused using an air-freshener device.

6. A method of preparing a pharmaceutical composition comprising the step of incorporating a compound of formula (I) according to claim 1 in the said pharmaceutical composition.

7. A compound of formula (Ia) 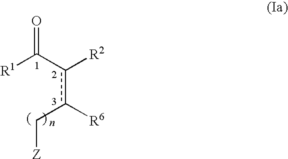
wherein n is 0 or 1 ;
the dotted line represents together with the carbon-carbon bond a double bond, either in E or Z configuration, or a single bond;
R¹ is C₁-C₃ alkyl, C₃-C₇ alkenyl, cycloalkylvinyl comprising from 5 to 7 carbon atoms, arylvinyl comprising from 5 to 7 carbon atoms, phenyl, C₁-C₃ alkoxy, C₂-C₃ alkenyloxy, or ethinyl;
R² is linear or branched C₃-C₇ alkyl;
R⁶ is H, C₁-C₃ alkyl, or —CH₂— forming with C-2 a cyclopropan ring;
I) Z is —CR³R⁴R⁵ wherein R³, R⁴, R⁵ are hydrogen, R³ and R⁴ are methyl and R⁵ is hydrogen or methyl; or R³ and R⁴ representing independently H, or C₁-C₆ alkoxy and R⁵ is C₁-C₆ alkoxy;
with the proviso that if n is 0, R² is n-pentyl and R¹ is methyl, Z is not prop-2-yl;
II) Z is a 3-6 membered monocyclic or 6-10 membered bicyclic hydrocarbon ring wherein up to two C atom(s) are replaced by a hetero atom selected from S, O, and N;
with the proviso that
if n is 0, R² is linear C₃-C₅ alkyl and R¹ is methyl, Z is not phenyl;
if n is 0, R² is linear C₃-C₄ alkyl and R¹ is methyl, Z is not methoxyphenyl;
if n is 0, R² is n-pentyl and R¹ is methoxy, Z is not phenyl;
if n is 0, R² is n-hexyl, R¹ is methyl, and the carbon-carbon bond between C-2 and C-3 is a single bond, Z is not cyclopropyl;
III) Z is a 3-6 membered monocyclic or 6-10 membered bicyclic hydrocarbon ring wherein up to two C atom(s) are replaced by a hetero atom selected from S, O, and N, and the ring is substituted with up to 5 groups selected from hydroxyl, CN, halogen, mono-, di-, and trihalogenomethyl, C₁-C₃ alkoxy, C₁-C₃ alkyl and —COOR and —OCOR, wherein R is hydrogen, methyl, ethyl, propyl or isopropyl, with the proviso that the ring is substituted with up to one C₁-C₃ alkyl group;
IV) Z is a bivalent residue —CH₂—CH₂— forming together with the C-2 a cyclobutan and cyclopentan ring respectively; or
V) Z is —C(O)R⁷ wherein R⁷ is C₁-C₃ alkyl, or C₁-C₃ alkoxy;
and the compound of formula (Ia) contains at least 9 C-atoms;
with the proviso that the compound of formula (Ia) is not 3-ethylideneoctan-2-one or 3-(propan-2-ylidene)octan-2-one.

8. A compound of formula (I) according to claim 7 selected from the group consisting of: (E)-3-(cyclopropylmethylene)octan-2-one; (E)-3-(cyclopropylmethylene)heptan-2-one; (E)-3-(cyclopropylmethylene)nonan-2-one; (1E,4E)-1-cyclopropyl-4-(cyclopropyl-methylene)dec-1-en-3-one; (E)-3-benzylideneheptan-2-one; (E)-3-benzylideneoctan-2-one; (1E,4E)-4-benzylidene-1-phenylnon-1-en-3-one; (E)-3-benzylidenenonan-2-one; 3-phenylmethylheptan-2-one; 3-phenylmethyloctan-2-one; (E)-4-(2-acetylhept-1-enyl)-benzonitrile; (E)-3-(naphthalen-2-ylmethylene)octan-2-one; (E)-3-(thiophen-2-ylmethylene)octan-2-one; (E)-3-(furan-2-ylmethylene)octan-2-one; 3-((tetrahydrofuran-2-yl)methyl)octan-2-one; (E)-3-((tetrahydrofuran-3-yl)methylene)heptan-2-one; (E)-3-((tetrahydrofuran-3-yl)methylene)octan-2-one; 3-((tetrahydrofuran-3-yl)methyl)octan-2-one; (E)-3-(2,2-dimethoxyethylidene)heptan-2-one; (E)-3-(2,2-dimethoxyethylidene)-octan-2-one; 3-(2,2-dimethoxyethyl)octan-2-one; 3-(2-methoxyethyl)octan-2-one; (E)-3-(2-(2-methyl-1,3-dioxolan-2-yl)ethylidene)octan-2-one; 3-(2-(2-methyl-1,3-dioxolan-2-yl)ethyl)octan-2-one; 3-pentylheptane-2,6-dione; 1-(2-methyl-1-pentylcyclopropyl)-ethanone; methyl 1-pentylcyclopentanecarboxylate; 1-(1-pentylcyclopentyl)-ethanone; (E)-3-(cyclohexylmethylene)octan-2-one; (E)-3-(cyclohex-3-enylmethylene)octan-2-one; (E)-3-(cyclopentylmethylene)octan-2-one; (E)-3-(cyclobutylmethylene)octan-2-one; (E)-3-(2-fluorobenzylidene)octan-2-one; (E)-3-(3-fluorobenzylidene)octan-2-one; (E)-3-(4-fluorobenzylidene)octan-2-one; (E)-3-(2,6-difluorobenzylidene)octan-2-one; (E)-3-(2,4-difluorobenzylidene)octan-2-one; (Z)-3-(3,5-difluorobenzylidene)octan-2-one; (E)-3-(3,5-difluorobenzylidene)octan-2-one; (E)-3-(perfluorobenzylidene)octan-2-one; (E)-3-(2-methylbenzylidene)octan-2-one; (E)-3-(3-methylbenzylidene)octan-2-one; (E)-3-(4-methylbenzylidene)octan-2-one; (E)-3-(2-(trifluoromethyl)benzylidene)octan-2-one; (E)-3-(2-methoxybenzylidene)octan-2-one; (E)-3-(3-methoxybenzylidene)octan-2-one; (E)-3-(4-methoxybenzylidene)octan-2-one; (E)-3-(benzo[d][1,3]dioxol-5-ylmethylene)octan-2-one; (Z)-methyl 4-(2-acetylhept-1-enyl)benzoate; (E)-methyl 4-(2-acetylhept-1-enyl)benzoate; (E)-3-(thiophen-2-ylmethylene)octan-2-one; (E)-3-(pyridin-2-ylmethylene)octan-2-one; (E)-3-(pyridin-3-ylmethylene)octan-2-one; (E)-3-(pyridin-4-ylmethylene)octan-2-one; (E)-methyl 2-(cyclopropylmethylene)heptanoate; methyl 2-(cyclopropylmethyl)heptanoate; 3-(cyclopropylmethyl)octan-2-one; (E)-3-(1-phenylethylidene)octan-2-one; (E)-2-(cyclopropylmethylene)-1-phenylheptan-1-one; (E)-methyl 2-(2,2-dimethylpropylidene)heptanoate; (E)-2-(2,2-dimethylpropylidene)heptanoic acid; (E)-3-(2,2-dimethylpropylidene)octan-2-one; (E)-4-(cyclopropylmethylene)nonan-3-one; and (E)-4-benzylidenenonan-3-one.
